(19) United States
(12) Patent Application Publication
(10) Pub. No.: US 2026/0142006 A1
(43) Pub. Date: May 21, 2026

(54) APPARATUS AND METHODS FOR DETERMINING SENSITIVITY TO OSIMERTINIB

(71) Applicant: Zephyr AI, Inc., Dallas, TX (US)

(72) Inventors: Maayan BARON, Carlsbad, CA (US); Maya RAMCHANDRAN, El Cerrito, CA (US); Jeff SHERMAN, Washington, DC (US); Dillon TRACY, Champagne, IL (US); Emily VUCIC, Los Angeles, CA (US)

(21) Appl. No.: 19/113,680

(22) PCT Filed: Sep. 21, 2023
(86) PCT No.: PCT/US2023/074774

§ 371 Date: Mar. 20, 2025

Related U.S. Application Data
(60) Provisional application No. 63/376,600, filed on Sep. 21, 2022.
(60) Provisional application No. 63/502,905, filed on May 17, 2023.

(51) Int. Cl. G16H 20/10; G16B 20/20; G16B 40/20; G16H 50/70

(52) U.S. Cl. CPC G16H 20/10; G16B 20/20; G16B 40/20; G16H 50/70

(57) ABSTRACT

Disclosed herein are methods of determining whether a subject suffering from non-small cell lung cancer (NSCLC) is sensitive to treatment with osimertinib. In some embodiments, the method comprises obtaining an experimental matrix comprising a mutation status of at least one gene in an experimental sample associated with the subject, applying the obtained experimental matrix to a model, the model being based on a partitioning matrix comprising, for a plurality of reference samples, a mutation status of at least one gene and a sensitivity label for each of the plurality of reference samples indicating whether a corresponding reference subject is sensitive to osimertinib, wherein the at least one gene of the partitioning matrix corresponds to the at least one gene of the experimental matrix, and determining the subject as sensitive to treatment with osimertinib based on a result of the applied model.

### CROSS-REFERENCE TO RELATED APPLICATIONS

This application claims the benefit of U.S. Provisional Patent Application No. 63/376,600, filed on Sep. 21, 2022, and U.S. Provisional Patent Application No. 63/502,905, filed on May 17, 2023, the content of each of which is herein incorporated by reference in its entirety.

### BACKGROUND

Non-small cell lung cancer (NSCLC) is any type of epithelial lung cancer other than small cell lung cancer (SCLC). Common types of NSCLC include squamous cell carcinoma, large cell carcinoma, and adenocarcinoma, but there are several other types that occur less frequently. NSCLC may be detected through standard imaging mechanisms (e.g., CT scan, MRI, etc.). Once detected, cancerous tissue may be biopsied and genotyped for mutations in pathways linked to NSCLC. Clinicians may use the outcome of the biopsy and/or mutational status to determine a treatment strategy that may generally include resection of tumors, radiation, or treatment with one or more adjuvant chemotherapeutic agents.

A number of chemotherapeutic agents have been FDA approved for NSCLC but are limited to specific patient populations. For example, osimertinib, sold under the brand name TAGRISSO®, is a tyrosine kinase inhibitor that is FDA approved for treatment of NSCLC with certain epidermal growth factor receptor (EGFR) mutations, namely exon 19 deletions or exon 21 L858R mutations. It may be the case however, that individuals suffering from NSCLC but who do not carry the specified EGFR mutations may also derive benefit from treatment with osimertinib. Provided herein are methods determining whether individuals suffering from NSCLC may respond to treatment with osimertinib.

### SUMMARY

According to embodiments, the present disclosure relates to apparatus, methods, and computer readable medium comprising instructions for determining whether a subject suffering from NSCLC is sensitive to treatment with osimertinib.

In some embodiments, the present disclosure further relates to a method for determining whether a subject suffering from non-small cell lung cancer (NSCLC) is sensitive to treatment with osimertinib, comprising obtaining an experimental matrix comprising a mutation status of at least one gene in an experimental sample associated with the subject, applying the obtained experimental matrix to a model, the model being based on a partitioning matrix comprising, for a plurality of reference samples, a mutation status of at least one gene and a sensitivity label for each of the plurality of reference samples indicating whether a corresponding reference subject is sensitive to osimertinib, wherein the at least one gene of the partitioning matrix corresponds to the at least one gene of the experimental matrix, and determining the subject as sensitive to treatment with osimertinib, based on a result of the applied model. In some embodiments, the at least one gene comprises one or more genes selected from the group consisting of: BCL2 apoptosis regulator (BCL2), GLI family zinc finger 2 (GLI2), GLI family zinc finger 3 (GLI3), lysine demethylase 5A (KDM5A), MAX dimerization protein MGA (MGA), signal transducer and activator of transcription 6 (STAT6), and tumor protein P53 (TP53), the at least one gene comprises one or more genes selected from the group consisting of: BCL2 apoptosis regulator (BCL2), cyclin D3 (CCND3), capicua transcriptional repressor (CIC), colony stimulating factor 3 receptor (CSF3R), exosome endoribonuclease (DIS3), forkhead box P1 (FOXP1), Kruppel like factor 4 (KLF4), large tumor suppressor kinase 1 (LATS1), ribosomal protein S6 kinase A4 (RPS6KA4), and signal transducer and activator of transcription 6 (STAT6), the at least one gene comprises one or more genes selected from the group consisting of: at least one selected from the group consisting of: BCL2 apoptosis regulator (BCL2), cyclin D3 (CCND3), capicua transcriptional repressor (CIC), colony stimulating factor 3 receptor (CSF3R), exosome endoribonuclease (DIS3), forkhead box P1 (FOXP1), GLI family zinc finger 2 (GLI2), GLI family zinc finger 3 (GLI3), lysine demethylase 5A (KDM5A), Kruppel like factor 4 (KLF4), large tumor suppressor kinase 1 (LATS1), MAX dimerization protein MGA (MGA), ribosomal protein S6 kinase A4 (RPS6KA4), signal transducer and activator of transcription 6 (STAT6), and tumor protein P53 (TP53), each reference sample within the partitioning matrix can be either EGFR wild-type or EGFR mutated, the model is based on a weighted contribution of each gene, the model is a logistic regression model, the model is a decision tree, the experimental sample is at least one selected from the group consisting of: blood, serum, plasma, urine, bile, sputum, tissue, cerebrospinal fluid, bone marrow aspirate, breast milk, saliva, synovial fluid, swabs, stool, and bronchial fluid. is at least one selected from the group consisting of: blood, serum, plasma, urine, and tissue, the sensitivity label is based on a differential in progression free survival with respect to its constituent sets, the plurality of reference samples are associated with a corresponding plurality of reference subjects, and/or the method further comprises administering one or more doses of osimertinib to the subject when the subject is determined to be sensitive to osimertinib.

In some embodiments, the present disclosure further relates to a method of determining whether a subject suffering from non-small cell lung cancer (NSCLC) is sensitive to treatment with osimertinib, comprising applying an experimental matrix to a model, wherein the model is based on a partitioning matrix comprising, for a plurality of reference samples, a mutation status of at least one gene and a sensitivity label for each of the plurality of reference samples indicating whether a corresponding reference subject is sensitive to osimertinib, and wherein the experimental matrix comprises a mutation status of a corresponding at least one gene in an experimental sample associated with the subject; and determining the subject as sensitive to treatment with osimertinib based on a result of the applied model.

In some embodiments, the present disclosure further relates to a method of determining whether a subject suffering from non-small cell lung cancer (NSCLC) is sensitive to treatment with osimertinib, comprising obtaining a partitioning matrix comprising, for each reference sample within the partitioning matrix, a mutation status of a plurality of genes and a sensitivity label for each reference sample indicating whether a corresponding reference subject is sensitive to osimertinib, generating a model based on the obtained partitioning matrix, obtaining an experimental matrix comprising a mutation status of the plurality of genes in an experimental sample associated with the subject, applying the obtained experimental matrix to the model, and determining the subject as sensitive to treatment with osimertinib based on a result of the applied model.

In some embodiments, the present disclosure further relates to an apparatus for determining whether a subject suffering from non-small cell lung cancer (NSCLC) is sensitive to treatment with osimertinib, comprising a processor configured to obtain an experimental matrix comprising a mutation status of at least one gene in an experimental sample associated with the subject, apply the obtained experimental matrix to a model, the model being based on a partitioning matrix comprising, for a plurality of reference samples, a mutation status of at least one gene and a sensitivity label for each of the plurality of reference samples indicating whether a corresponding reference subject is sensitive to osimertinib, wherein the at least one gene of the partitioning matrix corresponds to the at least one gene of the experimental matrix, and determine the subject as sensitive to treatment with osimertinib based on a result of the applied model.

In some embodiments, the present disclosure further relates to a non-transitory computer-readable storage medium storing computer-readable instructions that, when executed by a computer, cause the computer to perform a method of determining whether a subject suffering from non-small cell lung cancer (NSCLC) is sensitive to treatment with osimertinib, comprising obtaining an experimental matrix comprising a mutation status of at least one gene in an experimental sample associated with the subject, applying the obtained experimental matrix to a model, the model being based on a partitioning matrix comprising, for a plurality of reference samples, a mutation status of at least one gene and a sensitivity label for each of the plurality of reference samples indicating whether a corresponding reference subject is sensitive to osimertinib, wherein the at least one gene of the partitioning matrix corresponds to the at least one gene of the experimental matrix, and determining the subject as sensitive to treatment with osimertinib based on a result of the applied model.

In some embodiments, the present disclosure further relates to a method of determining whether a subject suffering from non-small cell lung cancer (NSCLC) is sensitive to treatment with osimertinib, comprising generating a model based on a partitioning matrix comprising, for each reference sample within the partitioning matrix, a mutation status of at least one gene and a sensitivity label for each reference sample indicating whether a corresponding reference subject is sensitive to osimertinib, and applying an obtained experimental matrix to the model, the experimental matrix comprising a mutation status of the at least one gene in an experimental sample associated with the subject to generate a label for the experimental sample indicating whether the subject is sensitive to treatment with osimertinib.

### BRIEF DESCRIPTION OF THE DRAWINGS

FIG. 1 depicts a flow diagram of aspects of an exemplary method of the present disclosure.

FIG. 2A depicts a flow diagram of aspects of an exemplary method of the present disclosure.

FIG. 2B depicts a flow diagram of aspects of an exemplary method of the present disclosure.

FIG. 3 depicts a flow diagram of aspects of an exemplary method of the present disclosure.

FIG. 4A depicts a graph of publicly available data of progression free survival of NSCLC subjects treated on label with osimertinib (“TAGRISSO®”).

FIG. 4B depicts a graph of the publicly available data of progression free survival of NSCLC subjects treated on label with osimertinib after being partitioned into a first group that exhibits an increase in progression free survival in response to osimertinib or a second group that exhibits relatively shorter progression free survival in response to osimertinib, by a machine learning model disclosed herein.

FIG. 4C depicts a table of Cox proportional hazard ratios (HR) in log₂ scale and p-values reported for potential confounders of the data of FIG. 4B.

FIG. 4D depicts a boxplot of area under the drug response curve values for cell lines partitioned into a first group (sensitive) and the second group (non-sensitive) of cancer cell lines, by a machine learning model disclosed herein.

FIG. 5 depicts a schematic of an exemplary decision tree generated from partitioned data.

FIG. 6A depicts a table representative of a possible readout generated by the machine learning model partitioning subjects into the first group or the second group based on mutational status of one or more genes.

FIG. 6B depicts a schematic of a decision tree generated from the partitioned data of FIG. 4B, the decision tree configured to predict if a NSCLC subject may be sensitive or non-sensitive to treatment with osimertinib.

FIG. 7 depicts the label for TAGRISSO®.

FIG. 8 depicts a Kaplan-Meier plot showing corresponding overall survival (OS) measurements of patients predicted, according to methods of the present disclosure, to be sensitive vs. insensitive to ismertinib. NSCLC patients predicted to be sensitive experienced a 35-month increased median OS than the predicted less sensitive group (log-rank test p-value<10-5; HR=1.57 (1.43-1.78, 95% CI)).

FIG. 9 depicts a method for further validation of sensitivity predictions using patient-dervied xenograft (PDX) models (HuBase™, CrownBio®). Treatment naïve PDX tumors were subcutaneously implanted into female non-obese diabetic/severe combined immunodeficient (NOD/SCID) or BALB/c nude mice in triplicate for a total of 96 mice (n=48 treated; 48 vehicle-treated controls). Mice were assigned labels. After implanted tumors reached a size of 200 mm³, mice were randomized into treatment and control groups. Treated mice were dosed with 25 mg/kg/day of osimertinib for 3 weeks. Changes in tumor volume were calculated. For each PDX model, tumor growth for treatment groups and control groups was assessed.

FIG. 10A is a graphical representation of tumor response of each cohort of FIG. 9. Each cohort had distinct tumor responses to treatment with osimertinib. As expected, mice in the FDA OFF/ZEPHYR OFF labeled cohort showed disease progression after treatment with osimertinib. Likewise, mice in the FDA ON/ZEPHYR OFF labeled cohort showed a mix of complete responses and disease progression, and the FDA ON/ZEPHYR ON labeled cohort showed complete response at the time of evaluation. The final cohort, labeled FDA OFF/ZEPHYR ON, showed partial to complete response to treatment with osimertinib. This final cohort represents an expansion of the patient population that can be successfully treated with osimertinib, a cohort that is not captured by the existing FDA label. FIG. 10B is a graphical representation of the data of FIG. 10A separated according to the indicated labeled cohorts (x-axis) as a function of the % of the PDX tumor models.

FIG. 11 is a graphical representation of the results of gene expression characterization of the cohorts shown in FIG. 10A and FIG. 10B.

FIG. 12 depicts a Gene Set Enrichment Analysis (GSEA, see, e.g., Subramanian et al., PNAS (2005) 102 (43) 15545-15550) of the data depicted in FIG. 11 and identification of core sensitivity subnetworks for sensitive (left) or less sensitive (right) tumors.

### DETAILED DESCRIPTION

The term “a” or “an” refers to one or more of that entity, i.e., can refer to plural referents. As such, the terms “a,” “an,” “one or more,” and “at least one” are used interchangeably herein. In addition, reference to “an element” by the indefinite article “a” or “an” does not exclude the possibility that more than one of the elements is present, unless the context clearly requires that there is one and only one of the elements.

Throughout this application, the term “about” is used to indicate that a value includes the inherent variation of error for the device or the method being employed to determine the value, or the variation that exists among the samples being measured. Unless otherwise stated or otherwise evident from the context, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents.

Non-small cell lung cancer (NSCLC) is the most commonly diagnosed lung cancer. Most treatment regimens include resection of tumors located in the lung tissue followed by treatment with one or more chemotherapeutic agents. Historically, many cancers were treated with radiation or one or more general chemotherapeutic agents. However, due to advancements in technology, tumors may be profiled through DNA sequencing, allowing clinicians to select targeted therapies for each patient. For example, in NSCLC, activating mutations may occur in a receptor known as epidermal growth factor receptor (EGFR), a tyrosine kinase. These mutations may lead to activation of the pathway in the absence of the epidermal growth factor ligand. The EFG/EGFR pathway is an important signaling pathway that is involved in proliferation, migration, differentiation, apoptosis, gene expression regulation, and intercellular communication during development. Commonly occurring activating mutations within EGFR include a point mutation resulting in the substitution of leucine to arginine at codon 858 (L858R) and short in-frame deletions of exon 19.

Tyrosine kinase inhibitors (TKI) have demonstrated benefits for subjects having activating mutations in tyrosine kinase pathways, including increases in progression free and overall survival and a reduction in disease burden in treated subjects (e.g., the first-generation TKI imatinib). The first- and second-generation EGFR TKIs imatinib, erlotinib, gefitinib, and afatinib have been widely used for advanced NSCLC patients.

Of course, despite the initial high response rates, patients on EGFR TKIs will inevitably become resistant to treatment. Because second generation TKIs have shown limited efficacy in circumventing T790 M resistance to first generation TKIs, third-generation TKIs were developed. A defining characteristic of third-generation TKIs is a have significantly greater activity in EGFR mutant cells than in EGFR WT cells, making them mutant-selective. Third-generation TKIs include, among others, osimertinib (marketed under the name TAGRISSO®), nazartinib, olmutinib, mavelertinib, lazertinib, avitinib, and rociletinib. Though it is expected that any of the third-generation EGFR TKIs would perform similarly, currently, the only approved third-generation EGFR TKI in the United States is osimertinib. As such, it will be the focus of the remainder of this disclosure.

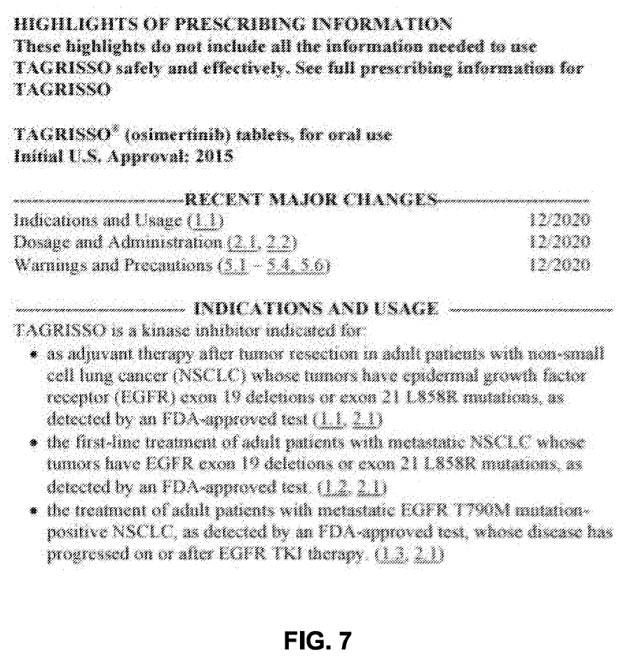

Osimertinib is FDA approved (“on-label”) to treat NSCLC for subjects having activating EGFR mutations including the short in-frame deletions in exon 19, the L858R substitution, or a T790M substitution (see FIG. 7). However, some NSCLC subjects lacking activating EGFR mutations designated as on-label may derive some benefit from treatment with osimertinib. Such a subject population is not currently positioned to receive osimertinib treatment, due to limitations of the label.

In order to undertake a label expansion approach, in view of e.g., the TAGRISSO® label shown in FIG. 7, publicly available real-world data, including sequence information and clinical outcome measures, are exploited by the method of the present disclosure. As a result, the present disclosure describes methods for determining whether a subject suffering from NSCLC is sensitive to treatment with osimertinib, wherein the determination is based on the mutational status of one or more of a plurality of possible genes. Mutational status determination includes, but is not limited to, detection of point mutations, additions, deletions, frame shifting mutations, translocations, and the like. The real-world data may include genomic sequencing data, gene expression data, RNA data, protein expression levels, age, gender, treatment regiment, geometric location, patient outcome, and the like, allowing the methods described herein to be applied without limits. The real-world data may include, for instance, sequencing data from subjects that have been clinically diagnosed with a NSCLC and have received an osimertinib regimen for that disease including on label FDA approved therapeutics or treatment through a clinical trial, or even off-label use.

The methods of the present disclosure are based on the application of the real-world data to a machine learning model that can be used to partition NSCLC subjects into two or more groups, wherein one group may derive benefit to or respond to treatment with osimertinib (“sensitive”) and one group may not derive benefit to or not respond to treatment with osimertinib (“non-sensitive”). The output of this model, referred to as a partition, is based on a mutation status of a plurality of genes within the sequencing data. For each subject within the real-world data, the mutational status of each gene within the sequencing data informs the machine learning models partitioning of the NSCLC subjects as being sensitive or not sensitive to osimertinib. In some embodiments, the plurality of genes may be related (e.g., in a common cellular pathway) or may be unrelated (e.g., in different cellular pathways).

Generally, the above-described machine learning model may be used in a method of generating a partition for a patient population diagnosed with NSCLC and treated with osimertinib. The method generally includes the steps of acquiring patient data, the patient data including but not limited to genomic sequencing data of patients within the patient population, applying the machine learning model to the acquired patient data, and generating the partition for the patient population based on the output of the machine learning model. The generated partition includes labels indicating whether or not each member of the patient population would derive some benefit from treatment with osimertinib (e.g., is sensitive to osimertinib, e.g., exhibits an improved clinical outcomes, such as increase in progression free survival). In some embodiments, the machine learning model may be trained on a corpus of reference data by methods known in the art, including supervised learning methods and unsupervised learning methods.

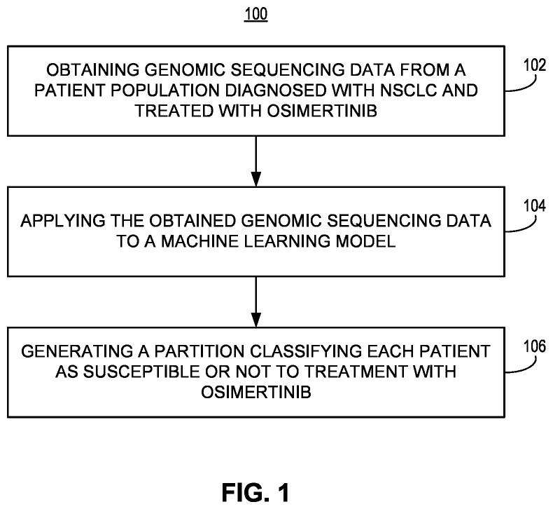

FIG. 1 depicts a flow chart of aspects of an exemplary method (100) of generating a partition classifying patients as either sensitive to treatment with osimertinib or not.

At step 102 of method 100, sequencing data (e.g., genomic sequencing data) is obtained from a patient population, or population of subjects, diagnosed with NSCLC and treated with osimertinib. The obtained sequencing data may include sequencing data from tumor cells, non-tumor cells, somatic cells, metastatic cells, or the like. In some embodiments, the obtained sequencing data may include providing sequencing data from the population of subjects before treatment with osimertinib and after treatment with osimertinib. The obtained sequencing data may include sequencing data from publicly available patient data sets, electronic medical record systems, medical insurance companies, commercial sequencing companies, health networks, or the like. In some embodiments, the sequencing data may be obtained from a population of subjects diagnosed with NSCLC and treated with osimertinib includes the genomic sequencing data having on-label EGFR mutations including EGFR exon 19 deletions, or the L858R or T790M substitutions (see FIG. 7). In some embodiments, the sequencing data may be obtained from the patient samples by laboratory diagnostics such as e.g., karyotyping, fluorescence in situ hybridization (FISH), comparative genomic hybridization, polymerase chain reaction (PCR), DNA microarray, DNA sequencing, multiplex ligation-dependent probe amplification, single strand conformation polymorphism, denaturing gradient gel electrophoresis, heteroduplex analysis, restriction fragment length polymorphism, and next-generation sequencing.

At step 104 of method 100, the obtained sequencing data may be applied to a machine learning model. The machine learning model may be previously trained according to supervised learning, unsupervised learning, reinforcement learning, or any derivative thereof (e.g., semi-supervised learning, self-supervised learning, multi-instance learning or the like). In some embodiments, the machine learning model may be a neural network such as a feed forward, multilayer perceptron, convolutional, radial basis functional, recurrent, long short-term memory, sequence to sequence models, modular, and the like.

At step 106 of the method 100, a partition is generated based on the output of the machine learning model. The partition classifies each patient within the patient population as sensitive or not to treatment with osimertinib. Patients classified as sensitive to treatment with osimertinib exhibit an increase in progression free survival in response to osimertinib compared to patients that are not sensitive.

In some embodiments, the partition generated by method 100 can be a matrix comprising, for each patient of the patient population, a mutational status of genes from the obtained sequencing data and a label generated for each patient of the patient population by the machine learning model, wherein the label identifies whether a particular patient is determined to be sensitive to treatment with osimertinib.

In some embodiments, the partition generated by method 100, or a reduced subset of partition 100, can be applied to corresponding genomic data from a new patient to identify whether the new patient is sensitive to treatment with osimertinib.

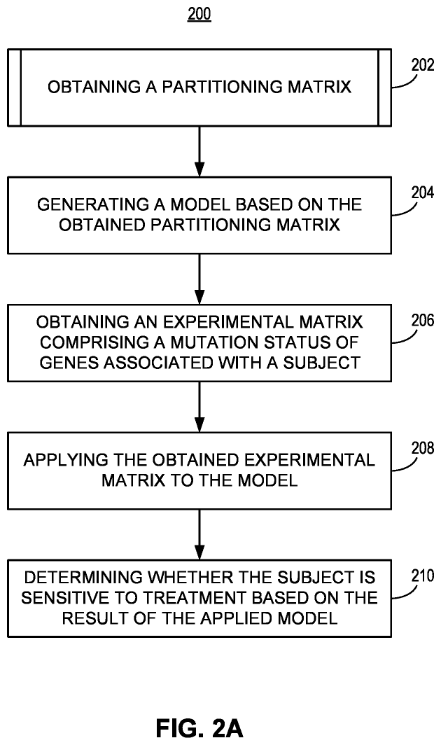

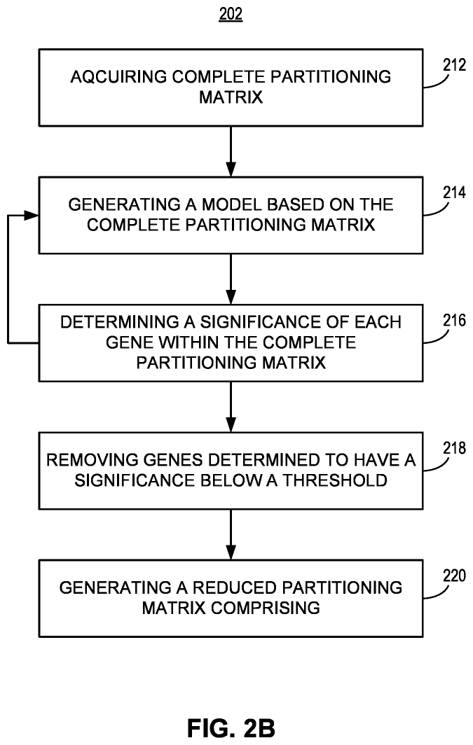

To this end, FIG. 2A and FIG. 2B are flow diagrams of aspects of implementation of the partition of method 100, in whole or in part, to a new patient. In particular, FIG. 2A provides a method 200 for determining whether a subject (e.g., new patient) is sensitive to treatment with osimertinib.

At subprocess 202 of method 200, a partitioning matrix can be obtained. In some embodiments, the partitioning matrix can be the partitioning matrix generated from method 100 of FIG. 1, wherein the partitioning matrix comprises, for each of a plurality of patients, mutational statuses for each of a plurality of genes and a sensitivity label indicating whether a respective patient is sensitive to osimertinib. The mutational status for each of the plurality of genes can be determined based on data derived from laboratory diagnostics for a sample obtained from each patient (within the partitioning matrix). Such diagnostics include, e.g., karyotyping, fluorescence in situ hybridization (FISH), comparative genomic hybridization, polymerase chain reaction (PCR), DNA microarray, DNA sequencing, multiplex ligation-dependent probe amplification, single strand conformation polymorphism, denaturing gradient gel electrophoresis, heteroduplex analysis, restriction fragment length polymorphism, and next-generation sequencing.

In some embodiments, the partitioning matrix obtained at subprocess 202 of method 200 is a complete partitioning matrix, comprising a complete set of the plurality of genes, and their mutational statuses, for each patient within the plurality of patients. Table 1 provides a set of genes within an exemplary complete partitioning matrix. In some embodiments, the plurality of genes comprises two or more, three or more, four or more, five or more, six or more, seven or more, eight or more, nine or more, or even ten or more of genes shown in Table 1.

TABLE 1
The plurality of genes within the complete partitioning matrix.

| | | | | | | |
|---|---|---|---|---|---|---|
| RAD50 | CDK4 | CDC73 | POT1 | H19 | DROSHA | PLK2 |
| ARID2 | SF3B1 | IKZF1 | NRG1 | PRDM1 | PAK1 | RPS6KB2 |
| FANCB | CBLB | BAP1 | LMO1 | DMC1 | INPPL1 | KNSTRN |
| SETD2 | AURKB | FANCE | MDM2 | MAX | PIK3C2G | SESN3 |
| POLD1 | GATA6 | RAD21 | MYCN | HMBS | GRIN2A | XIAP |
| EWSR1 | ALOX12B | NRAS | PPP2R1A | RAD51C | HLA-A | TAP2 |
| EXT1 | ARAF | SOX2 | SOS1 | SMAD2 | HIST1H3G | NCOR1 |
| EZH2 | ARID1B | CDH1 | DNASE1L1 | EPCAM | PARP1 | EIF4A2 |
| FLT1 | ZNF708 | CTD-2267D19.2 | BRCC3 | VEGFA | FOXP1 | WWTR1 |
| APC | BRIP1 | PALB2 | RBM10 | FH | MDC1 | NUF2 |
| NF1 | FGFR4 | STAG1 | SLX4 | TMEM127 | ANKRD11 | BTK |
| ROS1 | AXL | ID3 | RUNX1T1 | MAP2K2 | MALT1 | HIST3H3 |
| STK11 | TLR4 | KIT | CBFB | CXCR4 | DNMT1 | EGFL7 |
| TP53 | IGF1R | B2M | KDM5A | IL7R | SPEN | GATA1 |
| TSC1 | BUB1B | TERT | NSD1 | NEIL1 | MAP3K13 | RAD54L |
| ERCC3 | SQSTM1 | SMC1A | FOXL2 | ERG | FUBP1 | IGF1 |
| ERCC5 | DIS3 | EXT2 | ERCC6 | UBE2T | HIST1H3D | RAB35 |
| MDM4 | WRN | STAT6 | XRCC2 | RNF43 | TP63 | NTHL1 |
| NBN | KMT2A | CCND3 | HOXB13 | GBA | AMER1 | RXRA |
| RB1 | MET | ZNF217 | NR0B1 | TAL1 | DNMT3B | PIK3R3 |
| REL | MRE11A | NEGR1 | FAN1 | IGF2 | KMT2C | PPP4R2 |
| BRCA1 | RIF1 | FGFR2 | WHSC1L1 | JAZF1 | MAPK3 | MST1 |
| AKT3 | CSF3R | FLT3 | PTPN14 | MCM8 | EPHB1 | INHA |
| STAG2 | ERBB3 | MAPK1 | KAT6A | KLLN | INHBA | DNAJB1 |
| ASXL1 | FANCA | MAP2K4 | MLH3 | EGLN1 | TRAF2 | RRAS2 |
| ERBB2 | DICER1 | CBL | RPA1 | POLH | PRKD1 | ICOSLG |
| ERCC4 | MLH1 | CCND2 | TLX3 | MYCL | RAC1 | EIF1AX |
| FANCD2 | MUTYH | PRAME | TMPRSS2 | MAFB | DOT1L | E2F3 |
| GLI1 | NOTCH2 | COL7A1 | PNKP | IDH2 | PTPRT | H3F3C |
| PARK2 | PRKCI | FAT1 | TSHR | ID4 | INPP4A | CENPA |
| PDGFRA | SDHA | KIF1B | C17orf70 | RIT1 | PTPRS | FAM58A |
| PTPRD | TSC2 | CIC | EXO1 | AXIN2 | PDCD1 | H3F3B |
| RECQL4 | XPC | LMO2 | KCNQ1 | RAF1 | NCOA3 | RAD51B |
| CREBBP | EED | MAF | XRCC5 | CDK6 | HIST1H1C | RAC2 |
| GLI2 | RFWD2 | NOTCH3 | ABCB11 | NEIL2 | IRS2 | BBC3 |
| KRAS | ERCC2 | FANCI | KLF2 | GNA11 | HIST1H3I | STK19 |
| MAP3K1 | AR | RBBP8 | CBFA2T3 | RAD52 | TOP1 | CSDE1 |
| PIK3C2B | MPL | TET1 | ACVR1 | ERCC1 | JUN | SMAD3 |
| PBRM1 | DDR2 | MED12 | DCLRE1C | SMARCE1 | TGFBR2 | SESN1 |
| NFKBIZ | DDB2 | FGFR3 | BABAM1 | FAS | IRF4 | PIK3CD |
| ARID1A | CD274 | CDKN2B | MBD4 | XRCC3 | SOX17 | EPAS1 |
| KEAP1 | BCOR | FAM46C | STT3A | MUS81 | ERRFI1 | RPS6KA4 |
| CTNNB1 | GATA4 | XPO1 | EME1 | ABL1 | KMT2B | RTEL1 |
| EPHA3 | CIITA | CRKL | POLE | MTAP | ZFHX3 | FYN |
| ERBB4 | MITF | MYBL1 | RASA1 | NUTM1 | SMARCD1 | SHOC2 |
| MECOM | FUS | U2AF1 | SLC25A13 | GATA2 | TEK | SPRED1 |
| FANCC | PHF6 | PRPF40B | PVRL4 | PPARG | LATS2 | DUSP4 |
| FGFR1 | CSF1R | CHEK2 | POLQ | RNF8 | FGF19 | HIST1H1A |
| SETBP1 | BRD4 | ETV4 | LIG4 | XRCC6 | GSK3B | PDPK1 |
| SMAD4 | ALK | CCNE1 | SDHAF2 | CTLA4 | FOXO1 | CD79A |
| ATM | CADM2 | CDK5 | PTK2B | HELQ | TBX3 | AGO2 |
| ATRX | JAK3 | FANCF | RICTOR | TDG | BCL2L11 | HLA-B |
| PRKAR1A | TET2 | WT1 | SLC34A2 | YAP1 | STAT5B | DCUN1D1 |
| PRKDC | FLT4 | SDHB | ETV5 | PAXIP1-AS2 | PAK7 | LYN |
| BCORL1 | RET | CRLF2 | GEN1 | HIST1H3B | CD276 | BCL10 |
| SMARCA4 | RUNX1 | BMPR1A | SDHD | CDK8 | CUL3 | IKBKE |
| SMARCB1 | GNB2L1 | AKT2 | QKI | CTNNA1 | HGF | EIF4E |
| BRAF | KDM6B | HNF1A | RELA | BCL2 | PLCG2 | VTCN1 |
| AURKA | PAX5 | CDKN1A | PPM1D | RAD51 | HIST1H3E | SETD8 |
| KMT2D | SUFU | GNAS | CDKN2C | CD79B | IRS1 | RYBP |
| SLITRK6 | MAP2K1 | GNAQ | ELANE | FSBP | ELF3 | SESN2 |
| DEPDC5 | BCL6 | ZRSR2 | SERPINA1 | TCEB1 | SMYD3 | PPP6C |
| MEF2BNB-MEF2B | TCF3 | CEBPA | RAD51D | NAB2 | INSR | MSI2 |
| CDKN1B | MEN1 | TOPBP1 | VHL | POLB | PTP4A1 | CARM1 |
| CDKN2A | RPL26 | PAXIP1 | PHOX2B | SPOP | LATS1 | TNFRSF14 |
| PRPF8 | CDK2 | TRIM37 | KLF4 | SOCS1 | EZH1 | ABL2 |
| EGFR | GPC3 | UIMC1 | JAK1 | GALNT12 | ASXL2 | ACVR1B |
| EP300 | EPHA7 | FANCM | FANCL | NKX3-1 | IFNGR1 | GPR124 |
| EPHA5 | NFKBIA | SRSF2 | ARHGAP35 | SS18 | TGFBR1 | ARFRP1 |
| ESR1 | FBXW7 | TRAF7 | USP8 | CRTC2 | INPP4B | BTG1 |
| ETV1 | STAT3 | ENG | CYLD | SDHC | FGF4 | CHD4 |
| SUZ12 | KDM5C | FOXA1 | MYC | IKZF3 | YES1 | C11orf30 |
| MTOR | DNMT3A | IDH1 | FAH | XPA | RRAGC | FGF14 |
| GLI3 | PTK2 | CDK12 | RHOT1 | DMD | RRAS | FGF23 |
| MSH6 | TCF7L2 | USP28 | TP53BP1 | SYK | HIST1H3H | FGF6 |
| INSIG1 | MYB | CALR | HABP2 | H3F3A | BIRC3 | FRS2 |
| NOTCH1 | PTPN11 | CDH4 | NFKBIE | PSMD13 | STAT5A | GABRA6 |
| NTRK1 | DIS3L2 | SBDS | PGBD3 | PRKCZ | RHOA | GID4 |
| NTRK2 | SOX9 | PML | BRD3 | KCNIP1 | ARID5B | GRM3 |
| NTRK3 | WAS | ATR | HIST1H3C | RHEB | CYSLTR2 | HSD3B1 |
| PMS1 | XRCC1 | NEIL3 | BRE | PNRC1 | C1orf147 | HSP90AA1 |
| PTEN | RSPO3 | BARD1 | PRF1 | LMO3 | PGR | WDR20 |
| BLM | BCL2L12 | BCL2L1 | HFE | NPRL2 | PIK3C3 | IRF2 |
| SMO | TNFAIP3 | DKC1 | XRCC4 | CD58 | FGF3 | KEL |
| NKX2-1 | SF1 | AKT1 | UROD | NPRL3 | PRDM14 | LRP1B |
| KDM6A | CARD11 | KAT6B | RINT1 | NPM1 | STK40 | LZTR1 |
| RHPN2 | FKBP9 | DOCK8 | TRAF3 | PDCD1LG2 | SH2D1A | MAGI2 |
| SMC3 | CRTC1 | RHOH | CDKN1C | CDK1 | HIST1H3F | PAK3 |
| ETV6 | MSH2 | C9orf66 | MTA1 | CDK9 | FAM175A | PRSS8 |
| GNAS-AS1 | NF2 | CHEK1 | C1orf86 | SRC | HIST1H2BD | RP11-435O5.4 |
| HRAS | NFE2L2 | RSPO2 | MYD88 | PIM1 | UPF1 | RANBP2 |
| BRCA2 | PIK3CA | ITK | WHSC1 | CDKN2B-AS1 | AXIN1 | SLIT2 |
| KDR | TCF7L1 | RPTOR | CTCF | MST1R | HIST1H3J | SNCAIP |
| GATA3 | FANCG | BCL11B | DAXX | PREX2 | TAP1 | SOX10 |
| PDGFRB | CCND1 | TFE3 | CASP8 | RECQL | MAPKAP1 | SPTA1 |
| PMS2 | RARA | ZNRF3 | RHBDF2 | PIK3CG | GPS2 | STAT4 |
| JAK2 | GSTM5 | DDB1 | RAD54B | IL10 | MYOD1 | TAF1 |
| PTCH1 | RBL2 | NT5C2 | GREM1 | MSH3 | PIK3R2 | WISP3 |
| CUX1 | PIK3R1 | MGA | PRSS1 | NOTCH4 | SHQ1 | ZNF703 |
| SH2B3 | FLCN | ARHGEF12 | MCL1 | NUP93 | PIK3CB | |

In other embodiments, and as shown in FIG. 2B, the partitioning matrix obtained at subprocess 202 of method 200 is a reduced partitioning matrix generating based on the complete partitioning matrix but including those genes determined to significantly impact a sensitivity classification.

To this end, the complete partitioning matrix can be acquired at step 212 of method subprocess 202.

In a first iteration of step 214 of subprocess 202, a model of the complete partitioning matrix is generated. Model generation during the first iteration of step 214 of subprocess 202 is similar to model generation at step 204 of method 200. In some embodiments, the generated model may be one of a decision tree, a logistic regression model, or the like, and combinations thereof.

At step 216 of subprocess 202, a significance of each gene within the plurality of genes is determined. Genes determined to be significant are preserved within the final, reduced partitioning matrix and genes determined to be insignificant are discarded.

In some embodiments, determining the significance of each gene comprises evaluating a selected model, which may be a regression model. The regression model may be, in an example, one or more of a linear regression model, a logistic regression model, a ridge regression model, a LASSO regression model, a polynomial regression model, and a Bayesian linear regression model. Moreover, in some embodiments, determining the significance of each gene comprises performing statistical analyses on each gene within the selected model.

In some embodiments, wherein the model is a logistic regression model, performing statistical analyses may include generating the logistic regression model and determining whether a maximum likelihood estimation (MLE) falls within a multivariate normal distribution degree of confidence. It can be appreciated that that logistic regression model may identify any number of genes that may be significant to model performance. In some embodiments, the genes may include the presence or absence of a mutation within each gene of the plurality of genes, or the presence or absence of a specific mutation (e.g., activating, deletion, insertion, or the like).

In some embodiments, determining the significance of each gene comprises determining a coefficient for each gene by performing LASSO regression (Least Absolute Shrinkage and Selection Operator). LASSO regression is a regularization technique that is used over regression methods for a more accurate prediction and is based on shrinkage, where data values are shrunk towards a central point as the mean. Generally, the LASSO procedure encourages simple sparse models. In the present disclosure, the LASSO regression optimizes the classification task and, simultaneously, penalizes results that use too many genes (e.g., L1 penalty).

Steps 214 and 216 may be iteratively performed until each gene within the plurality of genes has been evaluated and identified as significant or not.

After identifying the significance of each gene with the plurality of genes, those genes determined to not be significant are removed, at step 218 of subprocess 202, and a reduced subset of genes, which hare significant, is formed.

At step 220 of subprocess 202, a reduced partitioning matrix is generated based on the reduced subset of genes.

In some embodiments, the size of the reduced partitioning matrix is further based on the model selected to be generated at step 214 of subprocess 202. Because the significance of each gene is determined according to the effect of that gene on the performance of the model, each type of model (e.g., decision tree, logistic regression, etc.) will require more or less genes and/or be impacted by more or less or different or same genes as other types of models. In further embodiments, appreciating that different model types can be selected, each model type generated may have minimal overlap with any other generated model type. Minimal overlap may include a minimal number of commonly shared genes between the models. A minimal number of commonly shared genes may include a defined range (e.g., about 1 to about 50) or a defined percentage (e.g., less than about % 1 to less than about 10%).

Table 2 provides an exemplary reduced partitioning matrix. In some embodiments, the reduced partitioning matrix comprises a reduced subset of genes and the reduced subset of genes comprises one or more, two or more, three or more, four or more, five or more, six or more, seven or more, eight or more, nine or more, ten or more, eleven or more, twelve or more, thirteen or more, fourteen or more, and/or fifteen or more of BCL2, CCND3, CIC, CSF3R, DIS3, FOXP1, GLI2, GLI3, KDM5A, KLF4, LATS1, MGA, RPS6KA4, STAT6, and TP53.

In some embodiments, the reduced subset of genes of the reduced partitioning matrix comprises one or more genes of the genes shown in Table 2.

TABLE 2

Gene
TP53
GLI2
GLI3
BCL2
STAT6
MGA
KDM5A

In some embodiments, and in view of NSCLC, genes downstream of EGFR may not be included within the reduced partitioning matrix. Likewise, the mutational statuses of each of the genes within the reduced partitioning matrix may be independent of any mutational statuses associated as drivers of the disease. It should also be appreciated that, in some embodiments, genes and respective mutational statuses of genes within the reduced partitioning matrix may not be independent of genes or pathways associated as drivers of the disease.

Returning now to FIG. 2A, a model may be generated at step 204 of method 200 based on the partitioning matrix obtained at subprocess 202 of the method 200, regardless of whether the obtained partitioning matrix is the complete partitioning matrix or the reduced partitioning matrix described in FIG. 2B.

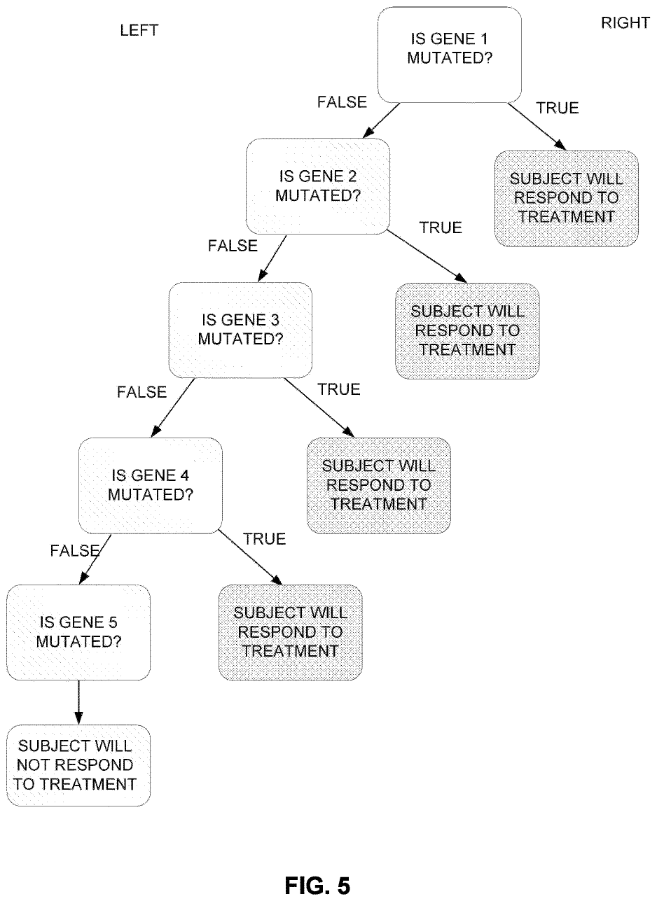

In some embodiments, the generated model may be a decision tree, as shown in FIG. 5. Following the steps decision tree of FIG. 5, which has been generated from a reduced partitioning matrix including only 10 genes for each patient, leads to a prediction of whether a subject may be sensitive to a therapeutic. In some embodiments, the decision tree model may include a plurality of split nodes wherein each split node is a test on a detected attribute. For example, in the decision tree of FIG. 5, each attribute is a known mutational status of a gene identified to be significant to a determination of whether a patient is sensitive to osimertinib. Each leaf node (i.e., TRUE determinations on the RIGHT; nodes that do not further split the data) represents the subject being classified as sensitive or not-sensitive to osimertinib. It can be appreciated that each split node may include tests on any attribute detected from the real-world data. It can be appreciated that the decision tree model may include any number of nodes.

In the decision tree of FIG. 5, each test (e.g., split node) within the decision tree may be based a binary determination of the mutational status of a relevant gene (e.g., whether or not the gene is mutated), while in other variations, each test may be based on a determination of a specific mutational status of the gene (e.g., activating mutation, deletion, substitution, or the like).

As shown in FIG. 5, a first node, or first split node, may include the test for the mutational status of Gene 1, wherein a TRUE outcome (indicated by an arrow towards the RIGHT) may indicate that the subject will be sensitive (e.g., respond to treatment) to treatment and a FALSE outcome (indicated by an arrow towards the LEFT) may indicate that additional attributes need to be tested. In this example, the TRUE outcome is a first leaf node. The second split node may include a test for the mutational status of Gene 2, wherein a TRUE outcome may indicate that the subject will be sensitive to treatment and a FALSE outcome may indicate that additional attributes need to be tested. The mutational status of each of Gene 3, Gene 4, and Gene 5 may be tested in the decision tree of FIG. 5 to determine whether a subject is sensitive to osimertinib.

In some embodiments, the model generated at step 204 of method 200 may be a logistic regression model configured to output a probability that a subject responds favorably to osimertinib. Considering the mutational status of each gene for the subject, the logistic regression model may determine the probability that the subject may be sensitive to the therapeutic.

For example, four genes (e.g., Gene 1, Gene 2, Gene 3, Gene 4) may be identified as important for predicting if a subject will be sensitive to treatment. Each of the four genes may include any identified gene and may be within the same canonical pathway or may be in different canonical pathways. Table 3 is a table of the four genes identified and their specific approximated weighted values (e.g., coefficient estimate). For example, the mutational statuses of each of the plurality of genes may be given a weighted value that may contribute to the probability of whether a subject may respond favorable to drug treatment. Mutations in some genes may contribute more significantly or less significantly to the subject responding to osimertinib.

TABLE 3
Identified Genes and Their Weighted Value

| Gene | Weighted Value |
|---|---|
| Gene 1 | 1.04 |
| Gene 2 | 0.36 |
| Gene 3 | −0.002 |
| Gene 4 | 0.012 |

A probability threshold may be used to classify the observation as either a 1 or a 0. In some variations, the probability threshold may be any number between about 0 and about 1, including about 0.3 to about 0.4, about 0.4 to about 0.5, about 0.5 to about 0.6, about 0.6 to about 0.7, about 0.7 to about 0.8, about 0.9 to about 1. Any value over the probability threshold, the model would predict the subject would be sensitive to osimertinib and any value below the probability threshold, the model would predict the subject would be non-sensitive to the therapeutic.

Returning now to FIG. 2A, the above-described model may benefit subjects or patients not yet approved for treatment with osimertinib but whose sequencing data demonstrate similar mutational statuses to subjects that demonstrate a high probability of sensitivity to treatment with osimertinib or have previously demonstrated sensitivity to treatment using osimertinib.

To this end, and in order to perform the determination of therapeutic sensitivity on e.g., a new patient, step 206 of method 200 includes obtaining an experimental matrix comprising a mutation status of genes associated with the new patient. The genes may correspond to those included within the complete partitioning matrix or reduced partitioning matrix from which the model was generated at step 204.

At step 208 of method 200, the experimental matrix can be applied to the generated model. Applying the experimental matrix may include solving for a sensitivity label of the new patient based on the generated model.

Accordingly, at step 210 of method 200, the output of the model can be used to determine whether the new patient is sensitive to osimertinib. In certain instances, the output of the model can be a probability, and the determination must be made on comparison of this probability to a threshold. In other embodiments, the output of the model may be a classification.

It can be appreciated that, in some embodiments, each of the mutational statuses of the genes comprise a different weighted value (as shown in Table 3) and applying the experimental matrix to the model may consider each weighted value of each mutational status when outputting a probability or determination regarding whether the new patient may respond to osimertinib. Responding to treatment with osimertinib may include an improved outcome as measured by an increase in progression free or overall survival when compared with non-responding patients.

In some embodiments, if the new patient is determined to respond to treatment with osimertinib at step 210, the method 200 further comprises administering the treatment to the new patient or generating instructions for administering the treatment to the new patient. Administering the treatment may include administering one or more doses of osimertinib to the subject for an appropriate amount of time or otherwise administering osimertinib as prescribed on the label.

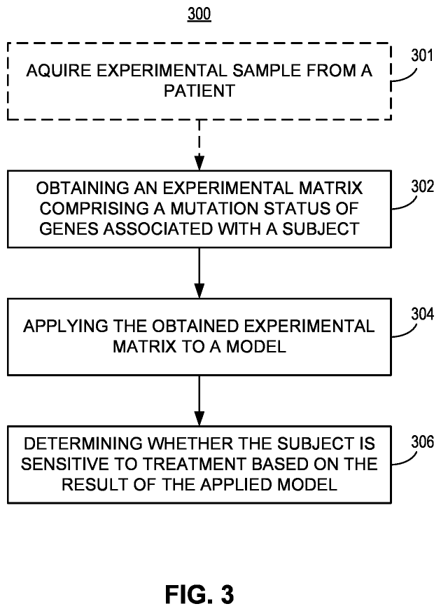

Referring now to FIG. 3, aspects of the methods described herein will be described. The flow diagram of FIG. 3 can be executed after model generation on the basis of a partitioning matrix (either complete or reduced).

At step 302 of method 300 of FIG. 3, an experimental matrix comprising sequencing data (e.g., genomic sequencing data) of a patient can be obtained. The genomic sequencing data may include mutational statuses of one or more genes corresponding to at least one gene within a corresponding model. The at least one gene within the corresponding model may refer to, in an instance, genes identified as significant to performance of a model that can be used to determine whether the patient is sensitive to osimertinib. In some embodiments, the mutational statuses for each of the one or more genes can be determined based on data derived from laboratory diagnostics for a sample obtained from the patient. Such diagnostics include but are not limited to karyotyping, fluorescence in situ hybridization (FISH), comparative genomic hybridization, polymerase chain reaction (PCR), DNA microarray, DNA sequencing, multiplex ligation-dependent probe amplification, single strand conformation polymorphism, denaturing gradient gel electrophoresis, heteroduplex analysis, restriction fragment length polymorphism, and next-generation sequencing.

In some embodiments, method 300 optionally includes step 301, wherein an experimental sample from the patient is acquired. The experimental sample may comprise any biological sample including but not limited to blood, serum, plasma, urine, bile, sputum, tumor samples, stool, pleural fluid, synovial fluid, CSF fluid, any tissues, organs, saliva, DNA/RNA, hair, nail clippings, or any other cells or fluids provided from a human body. In some embodiments, the experimental sample may be at least one selected from the group consisting of: blood, serum, plasma, urine, bile, sputum, tissue, cerebrospinal fluid, bone marrow aspirate, breast milk, saliva, synovial fluid, swabs, stool, and bronchial fluid. In some embodiments, the experimental sample may be from a patient that may or may not be presently diagnosed with NSCLC and may or may not have activating EGFR mutations (e.g., exon 19 deletions or exon 21 substitutions).

At step 304 of method 300, the obtained experimental matrix can be applied to a model. As noted above, the model may be based on a partitioning matrix comprising, for a plurality of patients, a sensitivity label and mutational statuses for a set of genes determined to be significant to the performance of the model.

At step 306, a sensitivity of the patient to osimertinib can be determined based on the output of the model.

In an example, wherein the patient is suspected of having NSCLC and osimertinib, the determination at step 306 may be that the patient is not sensitive to osimertinib.

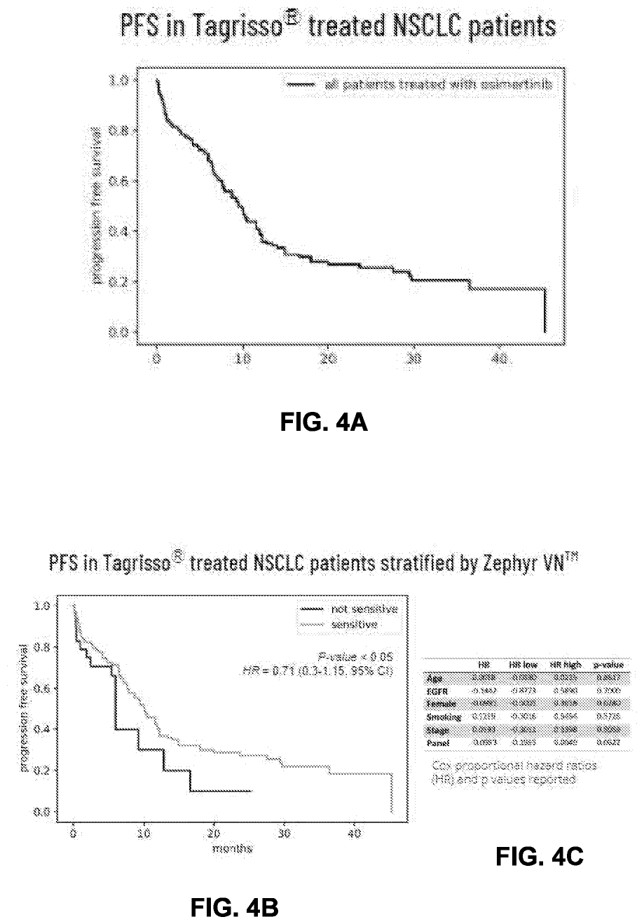

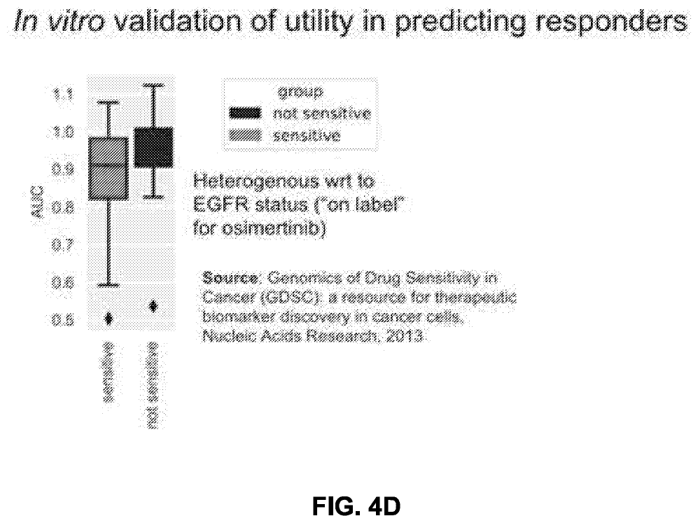

FIG. 4A through FIG. 4C depict a schematic, in view of FIG. 1, of generating a machine learning model capable of partitioning a population of subjects as either sensitive to treatment with osimertinib, or not sensitive to treatment with osimertinib. A population of NSCLC subjects having on-label EGFR mutations were treated with osimertinib. The entire population of NSCLC subjects was plotted on a graph depicting progression free survival against time (in months). Progression-free survival is typically defined and measured as the time from random assignment in a clinical trial to disease progression or death from any cause. In this schematic, random assignment in a clinical trial or real-world clinical setting includes the time starting treatment with osimertinib. Overall, the general trend of the subject population on the graph demonstrates that progression free survival decreases as time increases, even with treatment of osimertinib. However, within the subject population, some subjects may be responding favorably to treatment while other subjects may be responding negatively or not at all, leading to their disease progression or death, as time increases. Looking at the graph of the subject data, it is unclear which subjects responded favorably to treatment (indicated by a lack of a decrease in progression free survival over time or even an increase in progression free survival) and which subjects responded negatively or not at all (indicated by a decrease in progression free survival).

Using the trained machine learning model of FIG. 1, the data from the subjects may be analyzed and the corresponding subjects may be partitioned into a first group of subjects that do respond to treatment with osimertinib (sensitive) or a second group of subjects that do not respond to treatment with osimertinib (non-sensitive).

After partitioning of the subject population into the first group or the second group, the first group and the second group may be replotted on a graph depicting progression free survival against time (in months). The difference between the first group (sensitive) and the second group (non-sensitive) is clearly depicted, especially as time increases. The first group (sensitive) demonstrates a greater progression free survival compared to the second group (non-sensitive). Furthermore, the first group (sensitive) responds favorably to osimertinib treatment by demonstrating an increase in the duration of time the first group demonstrated a progression free survival. The partitioning of the NSCLC subject population demonstrates a powerful tool for clinicians to ensure their subjects are receiving proper care and therapeutic companies in designing therapeutics.

### Examples

The disclosure will now be illustrated with working examples, which is intended to illustrate the working of disclosure and not intended to take restrictively to imply any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein. It is to be understood that this disclosure is not limited to particular methods, and experimental conditions described, as such methods and conditions may apply.

### Example 1: Partitioning NSCLC Patient Samples into a Sensitive Group and a Non-Sensitive Group Using Machine Learning

A publicly available data set of de-identified genomic records including genomic sequencing data and progression free survival data was collected from about 2,000 NSCLC subjects. About 224 subjects of the about 2,000 NSCLC subjects had on-label EGFR mutations (activating EGFR mutations in exon 19 or exon 21) and were treated with osimertinib. The progression free survival of each patient versus time was plotted on a graph (FIG. 4A). A machine learning model was trained using a subset of the osimertinib treated subject data and tested using a subset of the osimertinib treated subject data. The osimertinib treated subject data was partitioned by the machine learning model into a first group (sensitive) and a second group (non-sensitive) and plotted on a progression free survival graph (FIG. 4B). The first group (sensitive) was significantly different from the second group (non-sensitive) as indicated by a p-value of less than 0.05 when controlling for potential confounders. FIG. 4C depicts a table of Cox proportional hazard ratios (HR) in log₂ scale and p-values reported for the potential confounders including age of the patient, EGFR status, sex of the patient, stage of disease, whether the patient smoked or not, and the like. The table indicates that the potential confounders do not account for the differences between the first group (sensitive) and the second group (non-sensitive), suggesting other differences are responsible for the partitioning of the patient population.

### Example 2: Validation of the Machine Learning Model

FIG. 4D depicts a graph of a box and whisker plot of area under the drug response curve (AUC) values for the first group (sensitive) and the second group (non-sensitive) of cancer cell lines. AUC of 1 indicates that the growth of the tumor cells exposed to the osimertinib is equivalent to their growth without exposure to osimertinib indicating lack of sensitivity to osimertinib. The first group (sensitive) has an average AUC of about 0.9 while the second group (non-sensitive) has an average AUC of about 0.95, indicating that the learned partitioning of the subject population is accurate. Furthermore, instead of generating the machine learning model wherein the model learns a point estimate for each of the parameters of the partitioning matrix (e.g., two or more genes), the machine learning model may be generated in a Bayesian context, wherein the machine learning model learns the posterior parameter distribution for the parameters of the partitioning matrix, the posterior parameter distribution may be approximately gaussian. For example, when the parameters of the partitioning matrix include 7 distinct genes, the machine learning model may learn the posterior parameter distribution for 8 different parameters (e.g., 7 genes plus an intercept term). In some embodiments, the posterior parameter distribution may have a confidence region of about 90%. The graphical representation of the confidence region of the posterior parameter distribution may depict ellipse enclosing a high-density region.

### Example 3: Using the Partitioned Data to Generate a Logistic Regression Model to Predict the Probability that a Subject Sample is Sensitive or not-Sensitive to Treatment

As described above, one model that may be generated from the partitioned data may be a logistic regression model, wherein each variable is the mutational status at a gene detected within the partitioned data and each mutational status includes a weighted value (e.g., coefficient estimate). The logistic regression model may be used to predict the probability of a label subject sample as sensitive or non-sensitive, wherein the label subject includes an NSCLC subject treated with osimertinib. Table 4 lists each of the genes having a mutational status important for predicting the probability of a label subject sample as sensitive or non-sensitive including the weighted value for each gene. This model may be used to further partition a subject into a first group (sensitive) or a second group (non-sensitive).

TABLE 4
Genes and weight values important for predicting the probability
of a label subject sample as sensitive or non-sensitive.

| Gene | Weight |
|---|---|
| TP53 | 0.6225 |
| GLI2 | −0.5831 |
| GLI3 | −1.0159 |
| BCL2 | −1.3179 |
| STAT6 | −3.3067 |
| MGA | −1.1311 |
| KDM5A | −2.0635 |

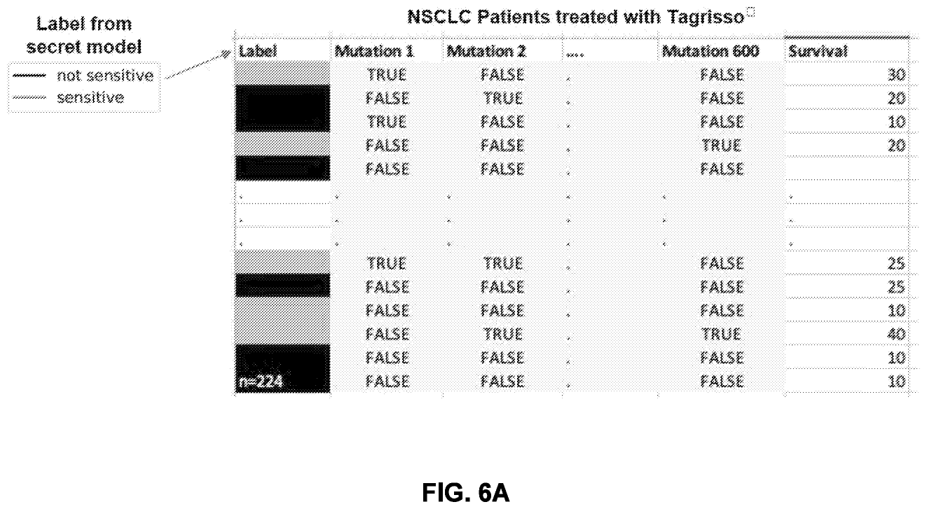

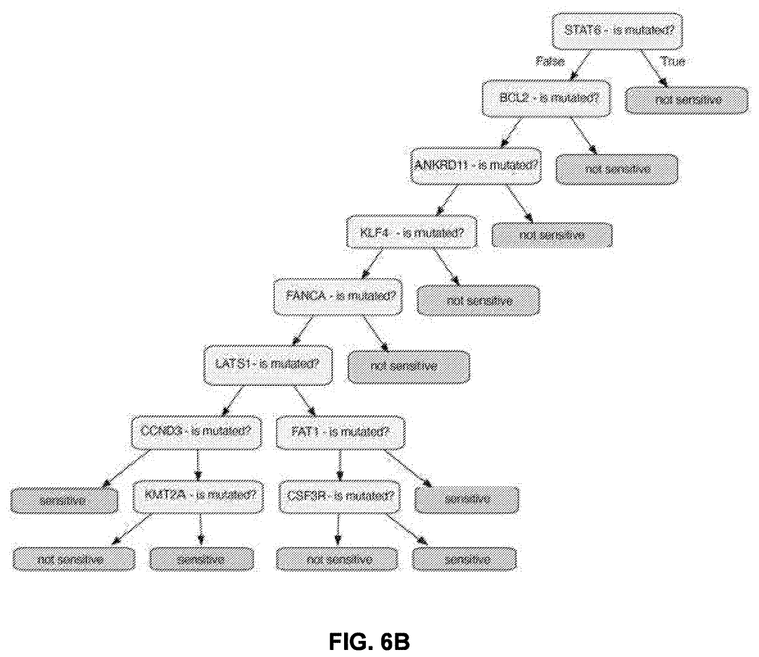

In partitioning the subject data into the first group or the second group, each of the mutational statuses may be given equal weight or a weighted value (e.g., each mutational status is equally important to the partitioning of the first group and second group) or unequal weight or a weighted value (e.g., some mutational statuses are more important than others in a particular partitioning of the first group and second group). The weighting of each of the mutational statuses may be determined by the machine learning model. In partitioning a subject to the first group (sensitive) or the second group (non-sensitive), the machine learning model may generate a readout of the label of the subject (e.g., sensitive, or non-sensitive) along with each of the mutational statuses of the genes comprising the partitioning matrix. FIG. 6A depicts a table of a readout generated by the machine learning model including a few subjects. The mutational status of each gene within the partitioning matrix may be listed in a column and each row may comprise the readout from an individual subject. As indicated in this table, the mutational status (e.g., presence of a mutation) within a gene in the partitioning matrix does not necessarily partition that patient to the first group (sensitive) or the second group (non-sensitive). In the top row, the subject has been partitioned into the first group (sensitive) and has the presence of Mutation 1, while in the fourth row down from the top row, the subject has also been partitioned into the first group (sensitive) but does not have the presence of Mutation 1. In some embodiments, the overall profile of all the collective mutational statuses of all the genes within the partitioning matrix may partition the patient into the first group (sensitive) or the second group (non-sensitive).

### Example 4: Using a Decision Tree to Partition NSCLC Subject Samples into the First Group (Sensitive) and the Second Group (Non-Sensitive)

As described above, different models may be generated from the partitioned data. FIG. 6B depicts an exemplary decision tree generated from the partitioned data of FIG. 4B. The decision tree depicted in FIG. 6B includes 10 nodes wherein each node is a test of a mutational status in a gene identified from the partitioned data as important in determining if a subject may be sensitive or non-sensitive to treatment with osimertinib.

Advantageously, the decision tree includes many genes that are distinct from genes contained within the Table 2, indicating that the decision tree of FIG. 6B has minimal overlap in the model of Example 3. In each of the first 5 nodes, a true result indicates the subject will not be sensitive to treatment with osimertinib, allowing the subject to be partitioned into the second group (non-sensitive). A false result in each of the first 5 nodes indicates that additional nodes (e.g., mutational statuses of additional genes) must be determined to partition the subject into either the first group (sensitive) or the second group (non-sensitive).

As indicated in this decision tree, the presence of a mutation in any one of FAT1, CSF3R, or KMT2A partitioned the subject into the first group (sensitive), with the absence of a mutation in CCND3 also partitioning the subject into the first group. The absence of a mutation in any one or more STAT6, BCL2, ANKRD11, KLF4, FANCA, LATS1, KMT2A, FAT1, or CSF3R partitions the subject into the second group (non-sensitive).

### Example 5: NSCLC Patients Predicted to be Sensitive to Osimertinib have Longer Progression Free Survival

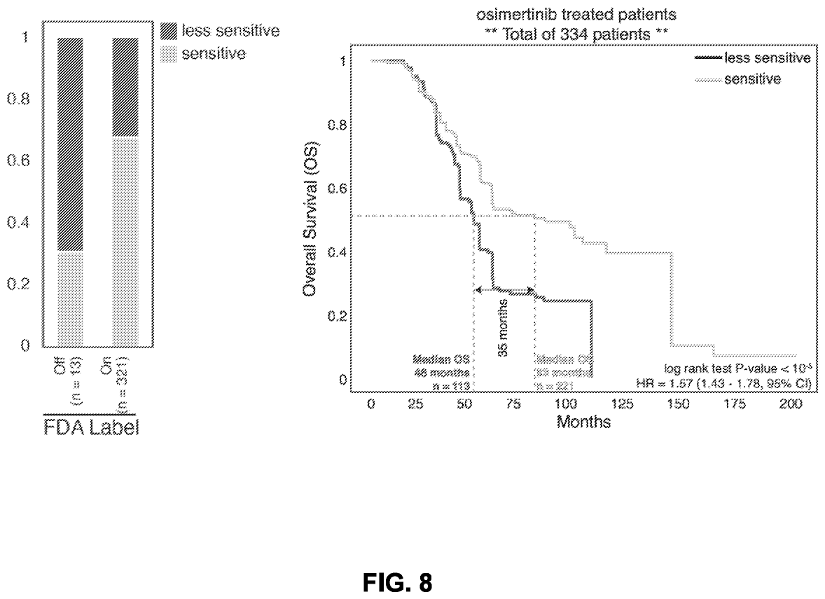

A clinicogenomics cohort from AACR Project GENIE, which included 334 patients treated with osimertinib, was used to validate the predictive ability of the methods disclosed herein. Data obtained in real-world settings were used to assign drug sensitivity predictions to individual patients in a RWD cohort of NSCLC patients treated with osimertinib. FIG. 8, a Kaplan-Meier plot, depicts overall survival (OS) measurements corresponding to patients predicted to be sensitive vs. insensitive. NSCLC patients predicted to be sensitive experienced a 35-month increased median OS than the NSCLC patients predicted to be less sensitive (log-rank test p-value<10-5; HR=1.57 (1.43-1.78, 95% CI)).

### Example 6. Preclinical Validation of the Predicted Response to Osimertinib and Demonstration of a Label Expansion Opportunity within NSCLC

To validate the predictions of tumor sensitivity/insensitivity, as described above, data were acquired using treatment-naïve NSCLC tumor (patient-derived xenograft) PDX models available via HuBase™ (CrownBio®, an online database offering complete genomic annotation of the PDX models).

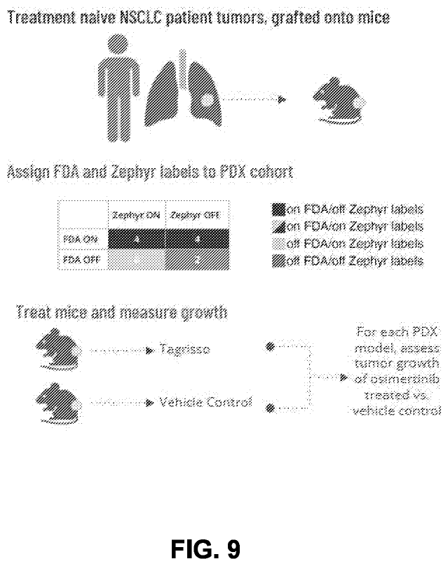

The current FDA label for osimertinib (FIG. 7) was used to assign PDX tumors to “FDA ON” or “FDA OFF” label groups based on the presence or absence of DNA alterations indicated for this drug (i.e., L858R mutation, T790M mutation, or exon 19 deletion). PDX tumors were also assigned label groups based on sensitivity predictions, as described above. Tumors labeled “Zephyr ON” included those that were predicted to be osimertinib-sensitive and samples labeled “Zephyr OFF” include those predicted to be osimertinib-insensitive. PDX models were then divided into four cohorts: FDA ON/ZEPHYR ON; FDA OFF/ZEPHYR ON; FDA ON/ZEPHYR OFF; and FDA OFF/ZEPHYR OFF. The cohorts were processed as follows and as shown in FIG. 9.

Treatment naïve PDX tumors were subcutaneously implanted into female NOD/SCID or BALB/c nude mice in triplicate for a total of 96 mice (n=48 treated; 48 vehicle-treated controls). After implanted tumors reached a size of 200 mm³, mice were randomized into treatment and control groups. Treated mice were dosed with 25 mg/kg/day of osimertinib for 3 weeks. Changes in tumor volume were calculated as previously described, e.g., in Gao et al., Nat. Med. 21, 1318-1325 (2015). For each PDX model, tumor growth for treatment groups and control groups (vehicle only) was assessed.

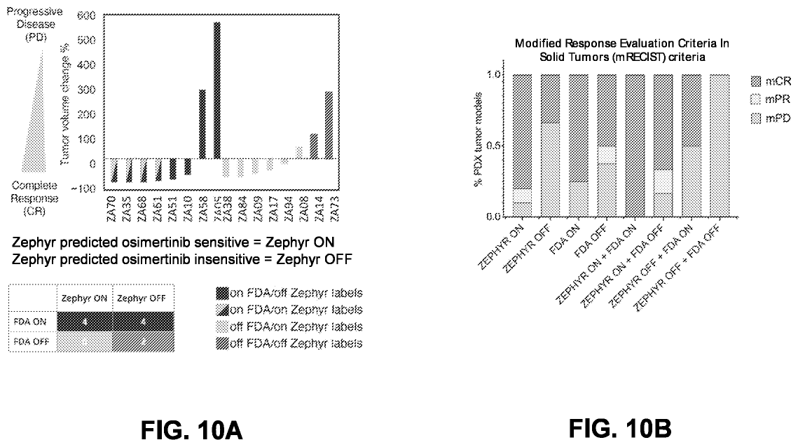

FIG. 10A and FIG. 10B show results in terms of Progressive Disease (mPD), Partial Response (mPR), and Complete Response (mCR) as evaluated by modified Response Evaluation Criteria in Solid Tumors (mRECIST) of samples from each of the four cohorts described above. As shown in FIG. 10A, each cohort had a distinct response to treatment with osimertinib. As expected, mice in the FDA OFF/ZEPHYR OFF cohort showed disease progression after treatment with osimertinib. Likewise, mice in the FDA ON/ZEPHYR OFF cohort showed a mix of complete responses and disease progression, and the FDA ON/ZEPHYR ON cohort showed complete response at the time of evaluation. The final cohort, FDA OFF/ZEPHYR ON, showed partial to complete response to treatment with osimertinib. This final cohort represents an expansion of the patient population that can be successfully treated with osimertinib that is not captured by the existing FDA label. FIG. 10B shows these same data separated according to the indicated groups (x-axis) as a function of the % of the PDX tumor models.

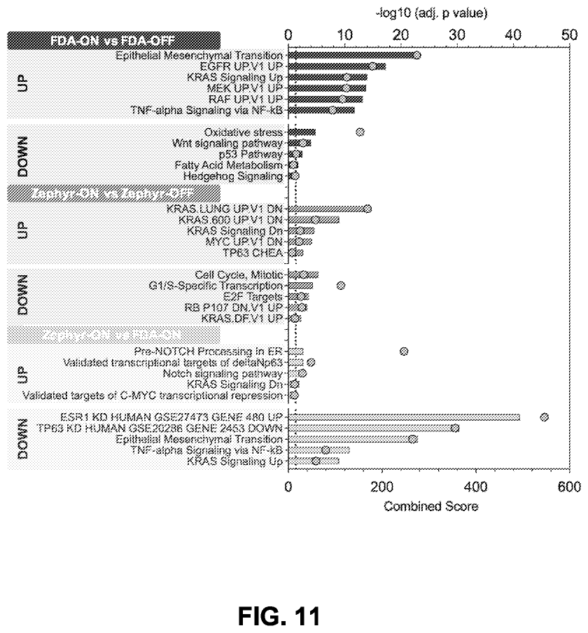

FIG. 11 is a graphical representation of results of a pathway-based gene expression characterization of the cohorts shown in FIG. 10A and FIG. 10B. The characterization includes FDA ON compared to FDA OFF (top), ZEPHYR ON compared to ZEPHYR OFF (middle), and ZEPHYR ON compared to FDA ON (bottom). Gene sets are either UP or DOWN as measured by the −log 10 adjusted p value. As expected, tumors covered by the FDA label and predicted as sensitive include EGFR-related pathways. In particular, the graph demonstrates that pathways enriched in label-expanded osimertinib-sensitive tumors (FDA OFF/ZEPHYR ON) indicate dependence on EGFR-related pathways, suggesting that a patient population exists that is not captured by the current FDA label for osimertinib. In other words, a population of osimertinib potential responders may exist whose tumors do not harbor EGFR mutations listed in the FDA label, which include the short in-frame deletions in exon 19, the L858R substitution, or a T790M substitution.

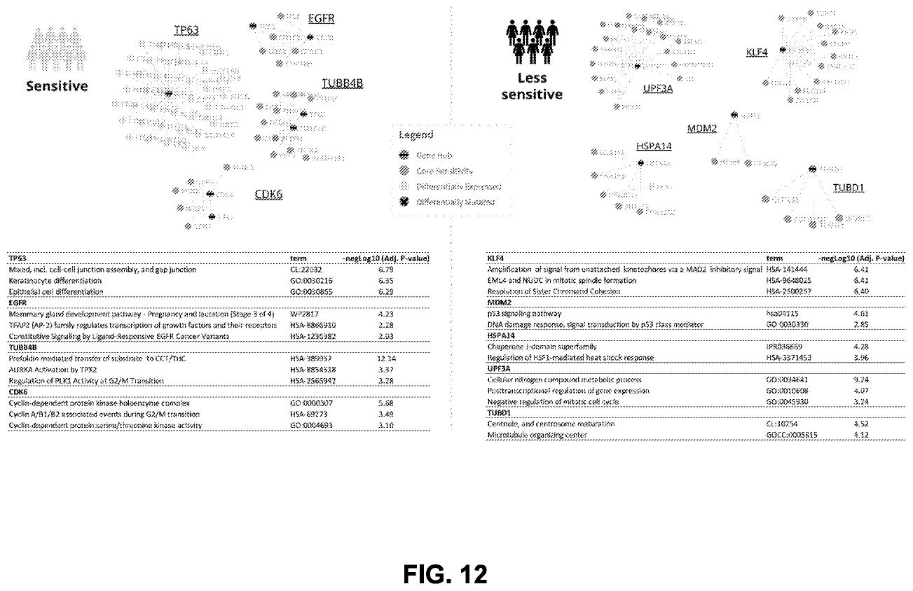

FIG. 12 is an illustration depicting a Gene Set Enrichment Analysis (GSEA, see, e.g., Subramanian et al., PNAS (2005) 102 (43) 15545-15550) of the data depicted in FIG. 11 and identification of core sensitivity subnetworks for sensitive (left) or less sensitive (right) tumors. Osimertinib-sensitive tumors harbor genetic sensitivity to EGFR perturbation but are not defined by EGFR mutations or TP63 and CDK6 genetic dependence.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

### Numbered Embodiments of the Invention

Notwithstanding the appended claims, the disclosure sets forth the following numbered embodiments:
(1) A method of determining whether a subject suffering from non-small cell lung cancer (NSCLC) is sensitive to treatment with osimertinib, comprising obtaining an experimental matrix comprising a mutation status of at least one gene in an experimental sample associated with the subject, applying the obtained experimental matrix to a model, the model being based on a partitioning matrix comprising, for a plurality of reference samples, a mutation status of at least one gene and a sensitivity label for each of the plurality of reference samples indicating whether a corresponding reference subject is sensitive to osimertinib, wherein the at least one gene of the partitioning matrix corresponds to the at least one gene of the experimental matrix, and determining the subject as sensitive to treatment with osimertinib based on a result of the applied model.
(2) The method of (1), wherein the at least one gene comprises one or more genes selected from the group consisting of: BCL2 apoptosis regulator (BCL2), GLI family zinc finger 2 (GLI2), GLI family zinc finger 3 (GLI3), lysine demethylase 5A (KDM5A), MAX dimerization protein MGA (MGA), signal transducer and activator of transcription 6 (STAT6), and tumor protein P53 (TP53).
(3) The method of either (1) or (2), wherein the at least one gene comprises one or more genes selected from the group consisting of: BCL2 apoptosis regulator (BCL2), cyclin D3 (CCND3), capicua transcriptional repressor (CIC), colony stimulating factor 3 receptor (CSF3R), exosome endoribonuclease (DIS3), forkhead box P1 (FOXP1), Kruppel like factor 4 (KLF4), large tumor suppressor kinase 1 (LATS1), ribosomal protein S6 kinase A4 (RPS6KA4), and signal transducer and activator of transcription 6 (STAT6).
(4) The method of any one of the preceding claims, wherein the at least one gene comprises one or more genes selected from the group consisting of: at least one selected from the group consisting of: BCL2 apoptosis regulator (BCL2), cyclin D3 (CCND3), capicua transcriptional repressor (CIC), colony stimulating factor 3 receptor (CSF3R), exosome endoribonuclease (DIS3), forkhead box P1 (FOXP1), GLI family zinc finger 2 (GLI2), GLI family zinc finger 3 (GLI3), lysine demethylase 5A (KDM5A), Kruppel like factor 4 (KLF4), large tumor suppressor kinase 1 (LATS1), MAX dimerization protein MGA (MGA), ribosomal protein S6 kinase A4 (RPS6KA4), signal transducer and activator of transcription 6 (STAT6), and tumor protein P53 (TP53).
(5) The method of any one of the preceding claims, wherein each reference sample within the partitioning matrix can be either epidermal growth factor receptor (EGFR) wild type or EGFR mutated.
(6) The method of any one of the preceding claims, wherein the model is based on a weighted contribution of each gene.
(7) The method of any one of the preceding claims, wherein the model is a logistic regression model.
(8) The method of any one of the preceding claims, wherein the model is a decision tree.
(9) The method of any one of the preceding claims, further comprising administering one or more doses of osimertinib to the subject when the subject is determined to be sensitive to osimertinib.
(10) The method of any one of the preceding claims, wherein the experimental sample is at least one selected from the group consisting of: blood, serum, plasma, urine, bile, sputum, tissue, cerebrospinal fluid, bone marrow aspirate, breast milk, saliva, synovial fluid, swabs, stool, and bronchial fluid.
(11) The method of (10), wherein the experimental sample is at least one selected from the group consisting of: blood, serum, plasma, urine, and tissue.
(12) The method of any one of the preceding claims, wherein the sensitivity label is based on a differential in progression free survival with respect to its constituent sets.
(13) The method of any one of the preceding claims, wherein the plurality of reference samples are associated with a corresponding plurality of reference subjects.
(14) A method of determining whether a subject suffering from non-small cell lung cancer (NSCLC) is sensitive to treatment with osimertinib, comprising applying an experimental matrix to a model, wherein the model is based on a partitioning matrix comprising, for a plurality of reference samples, a mutation status of at least one gene and a sensitivity label for each of the plurality of reference samples indicating whether a corresponding reference subject is sensitive to osimertinib, and wherein the experimental matrix comprises a mutation status of a corresponding at least one gene in an experimental sample associated with the subject, and determining the subject as sensitive to treatment with osimertinib based on a result of the applied model.
(15) A method of determining whether a subject suffering from non-small cell lung cancer (NSCLC) is sensitive to treatment with osimertinib, comprising obtaining a partitioning matrix comprising, for each reference sample within the partitioning matrix, a mutation status of a plurality of genes and a sensitivity label for each reference sample indicating whether a corresponding reference subject is sensitive to osimertinib, generating a model based on the obtained partitioning matrix, obtaining an experimental matrix comprising a mutation status of the plurality of genes in an experimental sample associated with the subject, applying the obtained experimental matrix to the model, and determining the subject as sensitive to treatment with osimertinib based on a result of the applied model.
(16) An apparatus for determining whether a subject suffering from non-small cell lung cancer (NSCLC) is sensitive to treatment with osimertinib, comprising a processor configured to obtain an experimental matrix comprising a mutation status of at least one gene in an experimental sample associated with the subject, apply the obtained experimental matrix to a model, the model being based on a partitioning matrix comprising, for a plurality of reference samples, a mutation status of at least one gene and a sensitivity label for each of the plurality of reference samples indicating whether a corresponding reference subject is sensitive to osimertinib, wherein the at least one gene of the partitioning matrix corresponds to the at least one gene of the experimental matrix, and determine the subject as sensitive to treatment with osimertinib based on a result of the applied model.
(17) The apparatus of (16), wherein the at least one gene comprises one or more genes selected from the group consisting of: BCL2 apoptosis regulator (BCL2), GLI family zinc finger 2 (GLI2), GLI family zinc finger 3 (GLI3), lysine demethylase 5A (KDM5A), MAX dimerization protein MGA (MGA), signal transducer and activator of transcription 6 (STAT6), and tumor protein P53 (TP53).
(18) The apparatus of either (16) or (17), wherein the at least one gene comprises one or more genes selected from the group consisting of: BCL2 apoptosis regulator (BCL2), cyclin D3 (CCND3), capicua transcriptional repressor (CIC), colony stimulating factor 3 receptor (CSF3R), exosome endoribonuclease (DIS3), forkhead box P1 (FOXP1), Kruppel like factor 4 (KLF4), large tumor suppressor kinase 1 (LATS1), ribosomal protein S6 kinase A4 (RPS6KA4), and signal transducer and activator of transcription 6 (STAT6).
(19) The apparatus of any one of (16) to (18), wherein the at least one gene comprises one or more genes selected from the group consisting of: at least one selected from the group consisting of: BCL2 apoptosis regulator (BCL2), cyclin D3 (CCND3), capicua transcriptional repressor (CIC), colony stimulating factor 3 receptor (CSF3R), exosome endoribonuclease (DIS3), forkhead box P1 (FOXP1), GLI family zinc finger 2 (GLI2), GLI family zinc finger 3 (GLI3), lysine demethylase 5A (KDM5A), Kruppel like factor 4 (KLF4), large tumor suppressor kinase 1 (LATS1), MAX dimerization protein MGA (MGA), ribosomal protein S6 kinase A4 (RPS6KA4), signal transducer and activator of transcription 6 (STAT6), and tumor protein P53 (TP53).
(20) The apparatus of any one of (16) to (19), wherein each reference sample within the partitioning matrix can be either epidermal growth factor receptor (EGFR) wild type or EGFR mutated.
(21) The apparatus of any one of (16) to (20), wherein the model is based on a weighted contribution of each gene.
(22) The apparatus of any one of (16) to (21), wherein the model is a logistic regression model.
(23) The apparatus of any one of (16) to (22), wherein the model is a decision tree.
(24) The apparatus of any one of (16) to (23), wherein the processing circuitry is further configured to administer one or more doses of osimertinib to the subject when the subject is determined to be sensitive to osimertinib.
(25) The apparatus of any one of (16) to (24), wherein the experimental sample is at least one selected from the group consisting of: blood, serum, plasma, urine, bile, sputum, tissue, cerebrospinal fluid, bone marrow aspirate, breast milk, saliva, synovial fluid, swabs, stool, and bronchial fluid.
(26) The apparatus of (25), wherein the experimental sample is at least one selected from the group consisting of: blood, serum, plasma, urine, and tissue.
(27) The apparatus of any one of (16) to (26), wherein the sensitivity label is based on a differential in progression free survival with respect to its constituent sets.
(28) The apparatus of any one of (16) to (27), wherein the plurality of reference samples are associated with a corresponding plurality of reference subjects.
(29) A non-transitory computer-readable storage medium storing computer-readable instructions that, when executed by a computer, cause the computer to perform a method of determining whether a subject suffering from non-small cell lung cancer (NSCLC) is sensitive to treatment with osimertinib, comprising obtaining an experimental matrix comprising a mutation status of at least one gene in an experimental sample associated with the subject, applying the obtained experimental matrix to a model, the model being based on a partitioning matrix comprising, for a plurality of reference samples, a mutation status of at least one gene and a sensitivity label for each of the plurality of reference samples indicating whether a corresponding reference subject is sensitive to osimertinib, wherein the at least one gene of the partitioning matrix corresponds to the at least one gene of the experimental matrix, and determining the subject as sensitive to treatment with osimertinib based on a result of the applied model.
(30) The non-transitory computer-readable storage medium of (29), wherein the at least one gene comprises one or more genes selected from the group consisting of: BCL2 apoptosis regulator (BCL2), GLI family zinc finger 2 (GLI2), GLI family zinc finger 3 (GLI3), lysine demethylase 5A (KDM5A), MAX dimerization protein MGA (MGA), signal transducer and activator of transcription 6 (STAT6), and tumor protein P53 (TP53).
(31) The non-transitory computer-readable storage medium of either (29) or (30), wherein the at least one gene comprises one or more genes selected from the group consisting of: BCL2 apoptosis regulator (BCL2), cyclin D3 (CCND3), capicua transcriptional repressor (CIC), colony stimulating factor 3 receptor (CSF3R), exosome endoribonuclease (DIS3), forkhead box P1 (FOXP1), Kruppel like factor 4 (KLF4), large tumor suppressor kinase 1 (LATS1), ribosomal protein S6 kinase A4 (RPS6KA4), and signal transducer and activator of transcription 6 (STAT6).
(32) The non-transitory computer-readable storage medium of any one of (29) to (31), wherein the at least one gene comprises one or more genes selected from the group consisting of: at least one selected from the group consisting of: BCL2 apoptosis regulator (BCL2), cyclin D3 (CCND3), capicua transcriptional repressor (CIC), colony stimulating factor 3 receptor (CSF3R), exosome endoribonuclease (DIS3), forkhead box P1 (FOXP1), GLI family zinc finger 2 (GLI2), GLI family zinc finger 3 (GLI3), lysine demethylase 5A (KDM5A), Kruppel like factor 4 (KLF4), large tumor suppressor kinase 1 (LATS1), MAX dimerization protein MGA (MGA), ribosomal protein S6 kinase A4 (RPS6KA4), signal transducer and activator of transcription 6 (STAT6), and tumor protein P53 (TP53).
(33) The non-transitory computer-readable storage medium of any one of (29) to (32), wherein each reference sample within the partitioning matrix can be either epidermal growth factor receptor (EGFR) wild type or EGFR mutated.
(34) The non-transitory computer-readable storage medium of any one of (29) to (33), wherein the model is based on a weighted contribution of each gene.
(35) The non-transitory computer-readable storage medium of any one of (29) to (34), wherein the model is a logistic regression model.
(36) The non-transitory computer-readable storage medium of any one of (29) to (35), wherein the model is a decision tree.
(37) The non-transitory computer-readable storage medium of any one of (29) to (36), further comprising administering one or more doses of osimertinib to the subject when the subject is determined to be sensitive to osimertinib.
(38) The non-transitory computer-readable storage medium of any one of (29) to (37), wherein the experimental sample is at least one selected from the group consisting of: blood, serum, plasma, urine, bile, sputum, tissue, cerebrospinal fluid, bone marrow aspirate, breast milk, saliva, synovial fluid, swabs, stool, and bronchial fluid.
(39) The non-transitory computer-readable storage medium of (38), wherein the experimental sample is at least one selected from the group consisting of: blood, serum, plasma, urine, and tissue.
(40) The non-transitory computer-readable storage medium of any one of (29) to (39), wherein the sensitivity label is based on a differential in progression free survival with respect to its constituent sets.
(41) The non-transitory computer-readable storage medium of any one of (29) to (40), wherein the plurality of reference samples are associated with a corresponding plurality of reference subjects.
(42) A method of determining whether a subject suffering from non-small cell lung cancer (NSCLC) is sensitive to treatment with osimertinib, comprising generating a model based on a partitioning matrix comprising, for each reference sample within the partitioning matrix, a mutation status of at least one gene and a sensitivity label for each reference sample indicating whether a corresponding reference subject is sensitive to osimertinib, and applying an obtained experimental matrix to the model, the experimental matrix comprising a mutation status of the at least one gene in an experimental sample associated with the subject to generate a label for the experimental sample indicating whether the subject is sensitive to treatment with osimertinib.
(43) The method of (42), wherein the at least one gene comprises one or more genes selected from the group consisting of: BCL2 apoptosis regulator (BCL2), GLI family zinc finger 2 (GLI2), GLI family zinc finger 3 (GLI3), lysine demethylase 5A (KDM5A), MAX dimerization protein MGA (MGA), signal transducer and activator of transcription 6 (STAT6), and tumor protein P53 (TP53).
(44) The method of either (42) or (43), wherein the at least one gene comprises one or more genes selected from the group consisting of: BCL2 apoptosis regulator (BCL2), cyclin D3 (CCND3), capicua transcriptional repressor (CIC), colony stimulating factor 3 receptor (CSF3R), exosome endoribonuclease (DIS3), forkhead box P1 (FOXP1), Kruppel like factor 4 (KLF4), large tumor suppressor kinase 1 (LATS1), ribosomal protein S6 kinase A4 (RPS6KA4), and signal transducer and activator of transcription 6 (STAT6).
(45) The method of any one of (42) to (44), wherein the at least one gene comprises one or more genes selected from the group consisting of: at least one selected from the group consisting of: BCL2 apoptosis regulator (BCL2), cyclin D3 (CCND3), capicua transcriptional repressor (CIC), colony stimulating factor 3 receptor (CSF3R), exosome endoribonuclease (DIS3), forkhead box P1 (FOXP1), GLI family zinc finger 2 (GLI2), GLI family zinc finger 3 (GLI3), lysine demethylase 5A (KDM5A), Kruppel like factor 4 (KLF4), large tumor suppressor kinase 1 (LATS1), MAX dimerization protein MGA (MGA), ribosomal protein S6 kinase A4 (RPS6KA4), signal transducer and activator of transcription 6 (STAT6), and tumor protein P53 (TP53).
(46) The method of any one of (42) to (45), wherein each reference sample within the partitioning matrix can be either epidermal growth factor receptor (EGFR) wild type or EGFR mutated.
(47) The method of any one of (42) to (46), wherein the model is based on a weighted contribution of each gene.
(48) The method of any one of (42) to (47), wherein the model is a logistic regression model.
(49) The method of any one of (42) to (48), wherein the model is a decision tree.
(50) The method of any one of (42) to (44), further comprising administering one or more doses of osimertinib to the subject when the subject is determined to be sensitive to osimertinib.
(51) The method of any one of (42) to (50), wherein the experimental sample is at least one selected from the group consisting of: blood, serum, plasma, urine, bile, sputum, tissue, cerebrospinal fluid, bone marrow aspirate, breast milk, saliva, synovial fluid, swabs, stool, and bronchial fluid.
(52) The method of (51), wherein the experimental sample is at least one selected from the group consisting of: blood, serum, plasma, urine, and tissue.
(53) The method of any one of (42) to (52), wherein the sensitivity label is based on a differential in progression free survival with respect to its constituent sets.
(54) The method of any one of (42) to (53), wherein the plurality of reference samples are associated with a corresponding plurality of reference subjects.
(55) A method of determining whether a subject suffering from non-small cell lung cancer (NSCLC) is sensitive to treatment with a third-generation epidermal growth factor receptor (EGFR) tyrosine kinase inhibitor (TKI), comprising obtaining an experimental matrix comprising a mutation status of at least one gene in an experimental sample associated with the subject, applying the obtained experimental matrix to a model, the model being based on a partitioning matrix comprising, for a plurality of reference samples, a mutation status of at least one gene and a sensitivity label for each of the plurality of reference samples indicating whether a corresponding reference subject is sensitive to the third-generation EGFR TKI, wherein the at least one gene of the partitioning matrix corresponds to the at least one gene of the experimental matrix, and determining the subject as sensitive to treatment with the third-generation EGFR TKI based on a result of the applied model.
(56) The method of (55), wherein the at least one gene comprises one or more genes selected from the group consisting of: BCL2 apoptosis regulator (BCL2), GLI family zinc finger 2 (GLI2), GLI family zinc finger 3 (GLI3), lysine demethylase 5A (KDM5A), MAX dimerization protein MGA (MGA), signal transducer and activator of transcription 6 (STAT6), and tumor protein P53 (TP53).
(57) The method of either (55) or (56), wherein the at least one gene comprises one or more genes selected from the group consisting of: BCL2 apoptosis regulator (BCL2), cyclin D3 (CCND3), capicua transcriptional repressor (CIC), colony stimulating factor 3 receptor (CSF3R), exosome endoribonuclease (DIS3), forkhead box P1 (FOXP1), Kruppel like factor 4 (KLF4), large tumor suppressor kinase 1 (LATS1), ribosomal protein S6 kinase A4 (RPS6KA4), and signal transducer and activator of transcription 6 (STAT6).
(58) The method of any one of (55) to (57), wherein the at least one gene comprises one or more genes selected from the group consisting of: at least one selected from the group consisting of: BCL2 apoptosis regulator (BCL2), cyclin D3 (CCND3), capicua transcriptional repressor (CIC), colony stimulating factor 3 receptor (CSF3R), exosome endoribonuclease (DIS3), forkhead box P1 (FOXP1), GLI family zinc finger 2 (GLI2), GLI family zinc finger 3 (GLI3), lysine demethylase 5A (KDM5A), Kruppel like factor 4 (KLF4), large tumor suppressor kinase 1 (LATS1), MAX dimerization protein MGA (MGA), ribosomal protein S6 kinase A4 (RPS6KA4), signal transducer and activator of transcription 6 (STAT6), and tumor protein P53 (TP53).
(59) The method of any one of (55) to (58), wherein each reference sample within the partitioning matrix can be either EGFR wild type or EGFR mutated.
(60) The method of any one of (55) to (59), wherein the model is based on a weighted contribution of each gene.
(61) The method of any one of (55) to (60), wherein the model is a logistic regression model.
(62) The method of any one of (55) to (61), wherein the model is a decision tree.
(63) The method of any one of (55) to (62), wherein the third-generation EGFR TKI is osimertinib, nazartinib, olmutinib, mavelertinib, lazertinib, or rociletinib.
(64) A method of determining whether a subject suffering from non-small cell lung cancer (NSCLC) is sensitive to treatment with a third-generation epidermal growth factor receptor (EGFR) tyrosine kinase inhibitor (TKI), comprising applying an experimental matrix to a model, wherein the model is based on a partitioning matrix comprising, for a plurality of reference samples, a mutation status of at least one gene and a sensitivity label for each of the plurality of reference samples indicating whether a corresponding reference subject is sensitive to the third-generation EGFR TKI, and wherein the experimental matrix comprises a mutation status of a corresponding at least one gene in an experimental sample associated with the subject, and determining the subject as sensitive to treatment with the third-generation EGFR TKI based on a result of the applied model.
(65) The method of (64), wherein the third-generation EGFR TKI is osimertinib, nazartinib, olmutinib, mavelertinib, lazertinib, or rociletinib.
(66) A method of determining whether a subject suffering from non-small cell lung cancer (NSCLC) is sensitive to treatment with a third-generation epidermal growth factor receptor (EGFR) tyrosine kinase inhibitor (TKI), comprising obtaining a partitioning matrix comprising, for each reference sample within the partitioning matrix, a mutation status of a plurality of genes and a sensitivity label for each reference sample indicating whether a corresponding reference subject is sensitive to the third-generation EGFR TKI, generating a model based on the obtained partitioning matrix, obtaining an experimental matrix comprising a mutation status of the plurality of genes in an experimental sample associated with the subject, applying the obtained experimental matrix to the model, and determining the subject as sensitive to treatment with the third-generation EGFR TKI based on a result of the applied model.
(67) The method of (66), wherein the third-generation EGFR TKI is osimertinib, nazartinib, olmutinib, mavelertinib, lazertinib, or rociletinib.
(68) An apparatus for determining whether a subject suffering from non-small cell lung cancer (NSCLC) is sensitive to treatment with a third-generation epidermal growth factor receptor (EGFR) tyrosine kinase inhibitor (TKI), comprising a processor configured to obtain an experimental matrix comprising a mutation status of at least one gene in an experimental sample associated with the subject, apply the obtained experimental matrix to a model, the model being based on a partitioning matrix comprising, for a plurality of reference samples, a mutation status of at least one gene and a sensitivity label for each of the plurality of reference samples indicating whether a corresponding reference subject is sensitive to the third-generation EGFR TKI, wherein the at least one gene of the partitioning matrix corresponds to the at least one gene of the experimental matrix, and determine the subject as sensitive to treatment with the third-generation EGFR TKI based on a result of the applied model.
(69) The apparatus of (68), wherein the third-generation EGFR TKI is osimertinib, nazartinib, olmutinib, mavelertinib, lazertinib, or rociletinib.
(70) A non-transitory computer-readable storage medium storing computer-readable instructions that, when executed by a computer, cause the computer to perform a method of determining whether a subject suffering from non-small cell lung cancer (NSCLC) is sensitive to treatment with a third-generation epidermal growth factor receptor (EGFR) tyrosine kinase inhibitor (TKI), comprising obtaining an experimental matrix comprising a mutation status of at least one gene in an experimental sample associated with the subject, applying the obtained experimental matrix to a model, the model being based on a partitioning matrix comprising, for a plurality of reference samples, a mutation status of at least one gene and a sensitivity label for each of the plurality of reference samples indicating whether a corresponding reference subject is sensitive to the third-generation EGFR TKI, wherein the at least one gene of the partitioning matrix corresponds to the at least one gene of the experimental matrix, and determining the subject as sensitive to treatment with the third-generation EGFR TKI based on a result of the applied model.
(71) The non-transitory computer-readable storage medium of (70), wherein the third-generation EGFR TKI is osimertinib, nazartinib, olmutinib, mavelertinib, lazertinib, or rociletinib.
(72) A method of determining whether a subject suffering from non-small cell lung cancer (NSCLC) is sensitive to treatment with a third-generation epidermal growth factor receptor (EGFR) tyrosine kinase inhibitor (TKI), comprising generating a model based on a partitioning matrix comprising, for each reference sample within the partitioning matrix, a mutation status of at least one gene and a sensitivity label for each reference sample indicating whether a corresponding reference subject is sensitive to the third-generation EGFR TKI, and applying an obtained experimental matrix to the model, the experimental matrix comprising a mutation status of the at least one gene in an experimental sample associated with the subject to generate a label for the experimental sample indicating whether the subject is sensitive to treatment with the third-generation EGFR TKI.
(73) The method of (72), wherein the third-generation EGFR TKI is osimertinib, nazartinib, olmutinib, mavelertinib, lazertinib, or rociletinib.

1. A method of determining whether a subject suffering from non-small cell lung cancer (NSCLC) is sensitive to treatment with osimertinib, comprising:
obtaining an experimental matrix comprising a mutation status of at least one gene in an experimental sample associated with the subject;
applying the obtained experimental matrix to a model, the model being based on a partitioning matrix comprising, for a plurality of reference samples, a mutation status of at least one gene and a sensitivity label for each of the plurality of reference samples indicating whether a corresponding reference subject is sensitive to osimertinib, wherein the at least one gene of the partitioning matrix corresponds to the at least one gene of the experimental matrix; and
determining the subject as sensitive to treatment with osimertinib based on a result of the applied model.

2. The method of claim 1, wherein the at least one gene comprises one or more genes selected from the group consisting of: BCL2 apoptosis regulator (BCL2), GLI family zinc finger 2 (GLI2), GLI family zinc finger 3 (GLI3), lysine demethylase 5A (KDM5A), MAX dimerization protein MGA (MGA), signal transducer and activator of transcription 6 (STAT6), and tumor protein P53 (TP53).

3. The method of claim 1, wherein the at least one gene comprises one or more genes selected from the group consisting of: BCL2 apoptosis regulator (BCL2), cyclin D3 (CCND3), capicua transcriptional repressor (CIC), colony stimulating factor 3 receptor (CSF3R), exosome endoribonuclease (DIS3), forkhead box P1 (FOXP1), Kruppel like factor 4 (KLF4), large tumor suppressor kinase 1 (LATS1), ribosomal protein S6 kinase A4 (RPS6KA4), and signal transducer and activator of transcription 6 (STAT6).

4. The method of claim 1, wherein the at least one gene comprises one or more genes selected from the group consisting of: at least one selected from the group consisting of: BCL2 apoptosis regulator (BCL2), cyclin D3 (CCND3), capicua transcriptional repressor (CIC), colony stimulating factor 3 receptor (CSF3R), exosome endoribonuclease (DIS3), forkhead box P1 (FOXP1), GLI family zinc finger 2 (GLI2), GLI family zinc finger 3 (GLI3), lysine demethylase 5A (KDM5A), Kruppel like factor 4 (KLF4), large tumor suppressor kinase 1 (LATS1), MAX dimerization protein MGA (MGA), ribosomal protein S6 kinase A4 (RPS6KA4), signal transducer and activator of transcription 6 (STAT6), and tumor protein P53 (TP53).

5. The method of claim 1, wherein each reference sample within the partitioning matrix can be either epidermal growth factor receptor (EGFR) wild type or EGFR mutated.

6. The method of claim 1, wherein the model is based on a weighted contribution of each gene.

7. The method of claim 1, wherein the model is a logistic regression model.

8. The method of claim 1, wherein the model is a decision tree.

9. The method of claim 1, further comprising:
administering one or more doses of osimertinib to the subject when the subject is determined to be sensitive to osimertinib.

10. The method of claim 1, wherein the experimental sample is at least one selected from the group consisting of: blood, serum, plasma, urine, bile, sputum, tissue, cerebrospinal fluid, bone marrow aspirate, breast milk, saliva, synovial fluid, swabs, stool, and bronchial fluid.

11. The method of claim 10, wherein the experimental sample is at least one selected from the group consisting of: blood, serum, plasma, urine, and tissue.

12. The method of claim 1, wherein the sensitivity label is based on a differential in progression free survival with respect to its constituent sets.

13. The method of claim 1, wherein the plurality of reference samples are associated with a corresponding plurality of reference subjects.

14-15. (canceled)

16. An apparatus for determining whether a subject suffering from non-small cell lung cancer (NSCLC) is sensitive to treatment with osimertinib, comprising:
a processor configured to
obtain an experimental matrix comprising a mutation status of at least one gene in an experimental sample associated with the subject,
apply the obtained experimental matrix to a model, the model being based on a partitioning matrix comprising, for a plurality of reference samples, a mutation status of at least one gene and a sensitivity label for each of the plurality of reference samples indicating whether a corresponding reference subject is sensitive to osimertinib, wherein the at least one gene of the partitioning matrix corresponds to the at least one gene of the experimental matrix, and
determine the subject as sensitive to treatment with osimertinib based on a result of the applied model.

17. The apparatus of claim 16, wherein the at least one gene comprises one or more genes selected from the group consisting of: BCL2 apoptosis regulator (BCL2), GLI family zinc finger 2 (GLI2), GLI family zinc finger 3 (GLI3), lysine demethylase 5A (KDM5A), MAX dimerization protein MGA (MGA), signal transducer and activator of transcription 6 (STAT6), and tumor protein P53 (TP53).

18. The apparatus of claim 16, wherein the at least one gene comprises one or more genes selected from the group consisting of: BCL2 apoptosis regulator (BCL2), cyclin D3 (CCND3), capicua transcriptional repressor (CIC), colony stimulating factor 3 receptor (CSF3R), exosome endoribonuclease (DIS3), forkhead box P1 (FOXP1), Kruppel like factor 4 (KLF4), large tumor suppressor kinase 1 (LATS1), ribosomal protein S6 kinase A4 (RPS6KA4), and signal transducer and activator of transcription 6 (STAT6).

19. The apparatus of claim 16, wherein the at least one gene comprises one or more genes selected from the group consisting of: at least one selected from the group consisting of: BCL2 apoptosis regulator (BCL2), cyclin D3 (CCND3), capicua transcriptional repressor (CIC), colony stimulating factor 3 receptor (CSF3R), exosome endoribonuclease (DIS3), forkhead box P1 (FOXP1), GLI family zinc finger 2 (GLI2), GLI family zinc finger 3 (GLI3), lysine demethylase 5A (KDM5A), Kruppel like factor 4 (KLF4), large tumor suppressor kinase 1 (LATS1), MAX dimerization protein MGA (MGA), ribosomal protein S6 kinase A4 (RPS6KA4), signal transducer and activator of transcription 6 (STAT6), and tumor protein P53 (TP53).

20. The apparatus of claim 16, wherein each reference sample within the partitioning matrix can be either epidermal growth factor receptor (EGFR) wild type or EGFR mutated.

21. The apparatus of claim 16, wherein the model is based on a weighted contribution of each gene.

22-28. (canceled)

29. A non-transitory computer-readable storage medium storing computer-readable instructions that, when executed by a computer, cause the computer to perform a method of determining whether a subject suffering from non-small cell lung cancer (NSCLC) is sensitive to treatment with osimertinib, comprising:
obtaining an experimental matrix comprising a mutation status of at least one gene in an experimental sample associated with the subject;
applying the obtained experimental matrix to a model, the model being based on a partitioning matrix comprising, for a plurality of reference samples, a mutation status of at least one gene and a sensitivity label for each of the plurality of reference samples indicating whether a corresponding reference subject is sensitive to osimertinib, wherein the at least one gene of the partitioning matrix corresponds to the at least one gene of the experimental matrix; and
determining the subject as sensitive to treatment with osimertinib based on a result of the applied model.

30-54. (canceled)
